SEQUENCE LISTING (15 sequences)

SEQ ID NO: 1 - DNA, 24 bp, Mus musculus
  Unsure (1)..(24)
  primer_bind (1)..(24)
aagagcgaccctcacatcaagcta

SEQ ID NO: 2 - DNA, 20 bp, Mus musculus
  unsure (1)..(20)
  primer_bind (1)..(20)
aagaaacactcatccgtaac

SEQ ID NO: 3 - DNA, 18 bp, mus musculus
  unsure (1)..(18)
  primer_bind (1)..(18)
ggaacaccgacaaaccca

SEQ ID NO: 4 - DNA, 18 bp, Mus musculus
  unsure (1)..(18)
  primer_bind (1)..(18)
tccccaaagcacagcaat

SEQ ID NO: 5 - DNA, 19 bp, Mus musculus
  unsure (1)..(19)
  primer_bind (1)..(19)
gggacggcaatgctgatgg

SEQ ID NO: 6 - DNA, 25 bp, Mus musculus
  Unsure (1)..(25)
  primer_bind (1)..(25)
tggtggcgcaccagcggtagccgtc

SEQ ID NO: 7 - DNA, 40 bp, Homo sapiens
  Unsure (1)..(40)
gtcgggccctatcccagtcccacttgctcgagcaagtggg

SEQ ID NO: 8 - DNA, 40 bp, homo sapien
  unsure (1)..(40)
gcgaattcaaaaagccctatcccagtcccacttgctcgag

SEQ ID NO: 9 - DNA, 22 bp, Homo sapiens
  unsure (1)..(22)
gggcccctggatggatagctac

SEQ ID NO: 10 - DNA, 21 bp, Homo sapiens
  unsure (1)..(21)
gtagctatccatccaggggcc

SEQ ID NO: 11 - DNA, 49 bp, Homo sapiens
  unsure (1)..(49)
gggcccctggatggatagctacctcgaggtagctatccatccaggggcc

SEQ ID NO: 12 - DNA, 54 bp, unknown
  AR siRNA with poly T after U6 promoter
tttttgggcccctggatggatagctacctcgaggtagctatccatccaggggcc

SEQ ID NO: 13 - DNA, 66 bp, unknown
  siRNA
gtcgggccctatcccagtcccacttgctcgagcaagtgggactgggatagggctttttgaattcgc

SEQ ID NO: 14 - protein, 919 aa, Homo sapiens
  UNSURE (1)..(919): AR protein sequence
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 15 - DNA, 4321 bp, Homo sapiens
  gene (1)..(4321)
cgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttagaattccggcggagagaaccctctgttttcccccactctctctccacctcctcctgccttccccaccccgagtgcggagcagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

(19) United States
(12) Patent Application Publication
(10) Pub. No.: US 2009/0282496 A1
(43) Pub. Date: Nov. 12, 2009

(54) Androgen Receptor Related Methods for Treating Bladder Cancer

(75) Inventor: Chawnshang Chang, Pittsford, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: University of Rochester Medical Center, Rochester, NY (US)

(21) Appl. No.: 12/418,369

(22) Filed: Apr. 3, 2009

Related U.S. Application Data
(60) Provisional application No. 61/042,707, filed on Apr. 4, 2008.

(51) Int. Cl. A01K 67/00; A61K 31/7088; A61K 39/395; A61K 31/12; A61K 31/16; A61K 31/165; C12N 15/63; C12Q 1/68; C12N 5/10

(52) U.S. Cl. 800/18; 514/44 R; 424/138.1; 514/683; 514/630; 514/618; 435/320.1; 435/6; 435/325

(57) ABSTRACT

Disclosed are compositions and methods for treating bladder cancer.

This application claims the benefit of U.S. Provisional Application No. 61/042,707, filed Apr. 4, 2008. This application is herein incorporated in its entirety.

### I. BACKGROUND

Men have a substantially higher risk of developing bladder cancer than women do (Jemal et al., CA Cancer J. Clin., 56:106-30 (2006)). Excessive exposure of men to cigarette smoke and industrial chemicals, both of which include amines, has been suggested to contribute to the development of bladder cancer (Hartge et al., J. Natl. Cancer Inst., 82:1036-40 (1990)). In the absence of exposure to known carcinogenic factors, there are sex-related differences in the risk of developing bladder cancer. (Hartge et al., J. Natl. Cancer Inst., 82:1036-40 (1990)). In experimental animal models, males are more likely than females to develop bladder cancer following exposure to certain chemical carcinogens (e.g., aromatic amines, such as N-butyl-N-(4-hydroxybutyl)nitrosamine [BBN]) (Bertram and Craig, Eur. J. Cancer, 8:587-94 (1972)). But other carcinogens, such as the arsenical metabolite dimethylarsinic acid, are more toxic to the female bladder than to the male bladder in rats (Shen et al., Toxicol. Appl. Pharmacol., 210:171-80 (2006)). This finding is consistent with epidemiologic evidence suggesting that women are more susceptible to arsenic-induced bladder cancer than are men (Smith et al., Environ. Health Perspect., 97:259-67 (1992)). In short, the bases for these sex-specific differences in the development of bladder cancer are not well understood.

### II. SUMMARY

Disclosed are methods and compositions related to Androgen Receptor and Bladder cancer.

In one aspect the disclosed methods and compositions related to the inhibition of the interaction of androgen and androgen receptor for the treatment of bladder cancer.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the chemical induction of bladder tumors in wild-type and androgen receptor knock-out (ARKO) mice. Six groups—1) male wild-type; 2) male castrated wild-type; 3) male ARKO; 4) male ARKO with DHT supplement; 5) female wild-type; and 6) female ARKO of mice—were treated with 0.05% BBN in drinking water, starting at 5-6 weeks of age. FIG. 1A shows representative lesions from a wild-type male mouse with invasive carcinoma (1,3) and an ARKO male mouse without a bladder tumor (2,4) both at 40 weeks of age are shown in macroscopic (1,2) and histopathologic (3,4; magnification=×100) views. In FIG. 1B, the left panels shown immunohistochemical analysis of proliferating cell nuclear antigen (PCNA) in representative bladder tumors harvested, at 40 weeks of age, from wild-type male mice (1; n=4), castrated male mice (2; n=4), wild-type female mice (3; n=4), and ARKO male mice with DHT supplement by slow-releasing pellet (1.5 mg/mouse/90 days) injection (4; n=3) (magnification=×400). Graph at right shows proliferation index, the percentage of PCNA-positive cells in 1000 cells in each tumor. Means and 95% confidence interval (CI) from 15 tumors are shown. In FIG. 1C, the left panels show fluorescence images generated by TUNEL analysis of the same bladder tumors as analyzed in (B) harvested from wild-type male mice (1; n=4), castrated male mice (2; n=4), wild-type female mice (3; n=4), and ARKO male mice with DHT supplement (4; n=3) (magnification=×400). Total cell population was visualized by staining with DAPI. Graph at right shows apoptotic index, the percentage of TUNEL-positive cells in 1000 cells in each tumor. Means and 95% CI from 15 tumors are shown.

FIG. 2 shows the effects of androgen and antiandrogen on bladder cancer cell lines. FIG. 2A shows that AR expression was analyzed in untreated bladder cancer cell lines (5637, 647V, J82, HT1197, HT1376, T24, TCC-SUP, and UMUC3) and prostate cancer cell lines (LNCaP, CWR22R, and PC3) by RT-PCR, and PCR products were separated on an agarose gel. PCR products derived from β-actin mRNA served as an internal control. FIG. 2B shows AR transactivation in bladder cancer cells. TCC-SUP or UMUC3 cells transfected with MMTV-Luc were cultured for 24 h in the presence of ethanol (ETOH), 1 nM DHT (androgen), and/or 5 μM HF (antiandrogen), as indicated, and luciferase activity was analyzed in a luminometer. Luciferase activity is presented relative to that of ETOH treatment in each cell line (set as 1-fold). Each value represents the mean and 95% confidence interval (CI) of at least three determinations. FIG. 2C shows bladder cancer cell proliferation. TCC-SUP or UMUC3 cells were cultured in the presence of ETOH, 1 nM DHT, 5 μM HF, and/or 1 μM ASC-J9 for 6 days, as indicated. Proliferation was assayed by MTT assay, and cell numbers are presented relative to cell number with ETOH treatment (set as 100%). Each value represents the mean and 95% CI of at least three determinations. FIG. 2D shows the growth of xenograft tumors in mice. TCC-SUP cells were implanted subcutaneously into the right and left flanks of male nude mice, and after 4 weeks, treatment (castration or sham surgery, injection of flutamide or placebo pellet, and/or intraperitoneal injection of ASC-J9 [50 mg/Kg dissolved in 10 μL DMSO+90 μL corn oil] or control [10 μL DMSO+90 μL corn oil] every other day) began. Tumor volume (n=12 tumors in each group, two tumors from each mouse) was monitored twice a week for 16 weeks (left panel). *1, castration group, p=0.0289; *2, ASC-J9 group, p=0.0201; *3, castration+flutamide group, p=0.0173; *4, flutamide group, p=0.0163 by repeated measures ANOVA test (vs. control group). The mice were then killed, and the tumors were harvested and weighed (right panel). #1, p=0.0395; #2, p=0.0250; #3, p=0.0167; #4, p=0.0445 by repeated measures ANOVA test (vs. control group). All the values represent the means and 95% CIs. FIG. 2E shows that the same tumor specimens (n=6 in each group, one tumor from each mouse) were analyzed for proliferation index (percentage of proliferating cell nuclear antigen-positive cells in 1000 cells), apoptotic index (percentage of TUNEL-positive cells in 1000 cells), and relative expression levels of AR, bFGF, VEGF, and MMP-9 as detected by real-time RT-PCR. For gene expression analyses, expression of each specific gene was normalized to that of □-actin. *1, p=0.0182; *2, p=0.0020; *3, p=0.0026; *4, p=0.0042; *5, p=0.0452; *6, p=0.0365; *7, p=0.0441; *8, p=0.0125; *9, p=0.0090; *10, p=0.0218; *11, p=0.0371; *12, p=0.0414; *13, p=0.0029; *14, p=0.0213; *15, p=0.0076; *16, p=0.0058; *17, p=0.0041 by t-test (vs. control group). All the values represent the means and 95% CIs of at least three determinations.

FIG. 3 shows the effect of AR-siRNA expression on bladder cancer cell growth in vitro and in vivo. FIG. 3A shows that bladder cancer cells (5637, an AR-negative line, and TCC-SUP and UMUC3, both AR-positive lines) were transfected with vectors containing either scrambled siRNA (control) or AR-siRNA. AR mRNA levels in stable transfectants were analyzed by RT-PCR (upper 2 lanes) and protein levels were analyzed by immunoblot (lower 2 lanes) analyses. FIG. 3B shows the scrambled control-siRNA- and AR-siRNA-transfected cells (one best subline for each combination) were cultured in the presence of ETOH, 1 nM DHT (androgen), and/or 5 μM HF (antiandrogen) for 6 days. Cell proliferation was assayed by MTT assay, and cell numbers are presented relative to cell number with ETOH treatment in each control line (set as 100%). Each value represents the mean and 95% CI of at least three determinations for each subline. (C) Scrambled control-siRNA and AR siRNA-transfected TCC-SUP and UMUC3 cells were implanted subcutaneously into the flanks (right: control-siRNA; left: AR-siRNA) of male nude mice. Treatment (castration or sham surgery, injection of flutamide or placebo pellet, and/or intraperitoneal injection of ASC-J9 [50 mg/Kg] or vehicle control [10 μL DMSO+90 μL corn oil] every other day) began at the time of tumor cell implantation. Tumor incidence (in n=8 mice in each group) was determined as positive when size reaches 40 mm3. Values represent the percentage of the mice, in each group, that developed tumors. *1, TCC-SUP/control-siRNA/castration, p=0.0166; *2, TCC-SUP/control-siRNA/flutamide, p=0.0459; *3, TCC-SUP/AR-siRNA/flutamide, p=0.0082; *4, TCC-SUP/AR-siRNA/castration, p=0.0088; *5, TCC-SUP/AR-siRNA/control, p<0.001; *6, TCC-SUP/control-siRNA/ASC-J9, p<0.001; *7, TCC-SUP/AR-siRNA/ASC-J9, p<0.001; *8, UMUC3/control-siRNA/flutamide, p=0.0485; *9, UMUC3/control-siRNA/castration, p=0.0258; *10, UMUC3/AR-siRNA/control, p=0.0116; *11, UMUC3/AR-siRNA/castration, p=0.0140; *12, UMUC3/AR-siRNA/flutamide, p=0.0250; *13, UMUC3/control-siRNA/ASC-J9, p=0.0038; *14, UMUC3/AR-siRNA/ASC-J9, p=0.0011 by Fisher's exact test (all comparisons versus the group of control-siRNA with control treatment).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Treating Cancer

The androgen receptor (AR) is a potential mediator of sex-specific differences in bladder cancer. The AR, a member of the nuclear receptor superfamily, is a ligand-dependent transcription factor that mediates the biologic effects of androgens (Chang et al., Science, 240:324-6 (1988) and Heinlein and Chang, Endocr. Rev., 25:276-308 (2004)). AR expression has been detected in normal bladder epithelium (Salmi et al., J. Urol., 166:674-7 (2001)) and in both male and female bladder carcinomas (Boorjian et al., Urology, 64:383-8 (2004)). But little is known about the role of AR in the bladder or the role of androgen metabolism in the bladder urothelium. Early studies showed that castration reduced the levels of cytochrome P450 CYP4B1, but that androgen supplementation helped these levels to recover (Imaoka et al., Cancer Lett., 166:119-23 (2001)). Cytochrome P450 CYP4B1 is predominantly present in the male bladder and activates amines to genotoxic substances. These findings raised the possibility that, by modulating the P450 system, androgens might affect the sex difference in incidence of BBN-induced bladder cancer in animals. In addition, the 5α-reductase inhibitor finasteride, which blocks the conversion of testosterone to dihydrotestosterone (DHT), only slightly suppressed the development of BBN-induced bladder cancer in rats, whereas castration had a much stronger effect (Imada et al., Eur. Urol., 31:360-4 (1997)). This difference suggests that DHT may not be more potent than testosterone at inducing bladder cancer. Because bladder cancer in humans is not generally considered to be dependent on hormone activity, previous studies in bladder cancer have not targeted the AR directly. Furthermore, neither androgen deprivation therapy, which is frequently used in prostate cancer treatment (Feldman and Feldman D, Nat. Rev. Cancer, 1:34-45 (2001); Chen et al., Nat. Med., 10:33-9 (2004); Miyamoto et al., Prostate, 61:332-53 (2004); Miyamoto et al., Nat. Clin. Prac. Oncol., 2:236-7 (2005)) nor other approaches that target the AR have been considered as therapeutic options for the treatment of human bladder cancer.

These epidemiologic and experimental observations indicate that androgens and/or the AR can play a role in development of bladder cancer. The ability of BBN to induce bladder cancer in wild-type and ARKO mice was first examined. Herein disclosed in one aspect is the role of AR signals in bladder cancer progression, AR-positive bladder cancer cell lines and mouse xenograft models were used in conjunction with either (1) androgen deprivation therapy, (2) modulation of AR activity by AR small-interfering RNA (siRNA), or (3) the anti-AR molecule ASC-J9.

Thus, for example, disclosed herein are methods of inhibiting uncontrolled cellular proliferation or treating cancer in a subject comprising administering to the subject an agent that inhibits one or more activities of androgen or the androgen receptor (AR). It is understood and herein contemplated that the inhibition can occur at the protein or genetic level. Thus, for example, disclosed herein are methods of inhibiting cell proliferation comprising administering the subject an agent that inhibits one or more activities of androgen or the androgen receptor gene. It is understood and herein contemplated that the one or more activities of an androgen or androgen receptor (AR) refers to any activity that the gene or protein is involved in such as signaling and binding a receptor or ligand. Thus, for example, activities of AR can include but is not limited to binding to androgen, signaling once ligand has bound, or providing a template for translation.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of bladder cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

A “decrease” can refer to any change that results in a smaller amount of a composition or compound, such as AR. Thus, a “decrease” can refer to a reduction in an activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a composition or compound, such as AR relative to a control. Thus, for example, an increase in the amount in AR can include but is not limited to a 10%, 20%, 30%, 40%, 50%, 60%, 70%, 80%, 90%, or 100% increase.

1. Androgen Receptor

Androgen receptor belongs to a superfamily of steroid hormone receptors and was first subcloned in 1988 (Chang, 1988). It contains an N-terminal transactivation domain, a central DNA binding domain (DBD) and a C-terminal ligand binding domain (LBD) (Umesono, 1995). By forming a homodimer and taking into account of the ligand and coregulators, the androgen receptors interact and regulate the transcription of numerous target genes (Ing, 1992; Schulman, 1995; Beatp, 1996; Yeh, 1996; Glass, 1997, Shibata, 1997). Besides its physiological roles, AR also contributes to pathological conditions highlighted by its role in prostate carcinogenesis (Quigley, C. A., et al. 1995. Endocr. Rev. 16:271-321, Santen, R. J. 1992. J. Clin. Endocrinol. Metab. 75:685-689). Like other members of SHR family, the AR contains an amino-terminal (N-terminal) transcription activation domain (TAD, amino acids 1-557 SEQ ID NO: 3 are AF1), a DNA-binding domain (DBD, amino acids 557-623), and a carboxyl-terminal ligand-binding domain (LBD, amino acids 624-919). (AF2 aa 872-908) (Mangelsdorf, D. J., et al., Cell 83:835-9 (1995)). Upon ligand binding, the AR dissociates from chaperone proteins including heat shock proteins, homodimerizes, translocates to the nucleus, and turns on the expression of its target genes by binding to the androgen receptor response element (ARE) (Quigley, C. A., et al. 1995. Endocr. Rev. 16:271-321; Chang, C., A. et al., Crit. Rev Eukaryot Gene Expr 5:97-125 (1995)). The strongest ligand of the androgen receptor is androgen. However, it is not the only ligand. Estradiol has been found to activate androgen receptor transactivation through the interaction with androgen receptor (Yeh, 1998). Also, androgen and androgen receptor do not only act in males. The increasing evidence has displayed that the androgen and androgen receptor (AR) may also play important role in female physiological processes, including the process of folliculogenesis, the bone metabolism and the maintenance of brain functions (Miller, 2001).

2. AR Domains

Compared to the quite conserved DBD and LBD, the N-terminus is quite polymorphic in terms of sequence and length between (nuclear receptors) NRs. The N-terminus is more likely to provide unique surfaces to recruit distinct factors that contribute to the specific action of a certain NR. The AR has a large N-terminus (ARN) and there are two distinct regions important for its transactivation function residing within the ARN: residues 141-338, which are required for full ligand-inducible transactivation, and residues 360-494, where the ligand-independent activation function-1 (AF-1) region is located (Heinlein, C. A., et al. 2002. Endocr. Rev. 23:175-200). Coactivators and corepressors have been identified to interact with ARN (Hsiao, P., et al. 1999. J. Biol. Chem. 274:22373-22379, Hsiao, P., et al. 1999. J. Biol. Chem. 274:20229-20234, Knudsen, K. E., et al. 1999. Cancer Res. 59:2297-2301, Lee, D. K., et al. 2000. J. Biol. Chem. 275:9308-9313, Markus, S. M., et al. 2002. Mol. Biol. Cell 13:670-682, Petre, C. E., et al. 2002, J. Biol. Chem., 277:2207-2215). Furthermore, although ARN extends to more than one half of the full length protein, its associated proteins are relatively fewer compared to those associated with AR DBD and AR LBD, presumably due to the existence of the AF-1 region which limits the application of conventional yeast-two hybrid system by using ARN as bait. It's likely there are still more ARN associated proteins remaining to be identified.

AR is classified with glucocorticoid receptor (GR), mineralocorticoid receptor and progesterone receptor (PR) as one group within the nuclear receptor (NR) superfamily, since they share high homology in the DBD and recognize very similar hormone response elements (Forman, B. M. et al. 1990. Mol. Endocrinol. 4:1293-1301, Laudet, V., et al. 1992. EMBO J. 11:1003-1013). However, the physiological responses mediated by these receptors upon cognate ligand activation are quite distinct and hormone specific. Apparently, these cannot be explained by a specific DNA-binding through the DBD. Factors located outside the DBD may play a key role in determining the specific hormone responses.

3. Coregulators Interact with AR and Other Steroid Receptors

Steroid receptors may function through direct or indirect interaction with other regulatory proteins in cells (McKenna, N. J., and B. W. 0'Malley, Cell 108:465-74 (2002); McKenna, N. J., and B. W. O'Malley, Endocrinology 143:2461-5 (2002)). There is a substantial amount of evidence to indicate that steroid hormone receptors function as a tripartite system, involving the receptor, its ligands, and its coregulator proteins (Katzenellenbogen et al. (1996) Mol. Endocrinol. 10, 119-131; Torchia et al. (1998) Curr. Opin. Cell Biol. 10, 373-383; McKenna et al. (1999) J. Steroid Biochem. Mol. Biol. 69, 3-12; Yeh et al. (1998) Proc. Natl. Acad. Sci. U.S.A. 95, 5524-5532; Miyamoto et al. (1998) Proc. Natl. Acad. Sci. U.S.A. 95, 7379-7384). A number of transcriptional coregulators, including coactivators and corepressors, have been identified that enhance or suppress the interactions between steroid receptors and the basal transcriptional machinery (Hermanson, O., et al., Trends Endocrinol Metab 13: 55-60 (2002); 31. Jepsen, K., et al., Cell 102:753-63 (2000); McInerney, E. M., et al., Proc Natl Acad Sci USA 93:10069-73 (1996); Xu, L., et al., Curr Opin Genet Dev 9:140-7 (1999)). These include ARA70, ARA55, ARA54, ARA24, ARA160, Rb, BRCA1, Smad3, AIB1 and SRC1. It has been suggested that regulation by coregulators is an efficient way to achieve cell- and promoter-specific activation (Pearce, D. et al. 1993. Science 259:1161-1164). A large number of coregulators have been identified in recent years (reviewed in Heinlein, C. A., et al. 2002. Endoer. Rev. 23:175-200, McKenna, N. J., et al. 1999. Endocr. Rev. 20:321-344). For example, SRC-1 can serve as a coactivator to many NRs like PR, estrogen receptor (ER), GR, thyroid hormone receptor (TR) and retinoid X receptor (RXR) (Onate, S. A., et al., Science 270:1354-1357 (1995)). Although NCo-R and SMRT were initially identified to mediate active suppression by unliganded TR and retinoid acid receptor (Chen, J. D., et al. 1995. Nature 377:454-457, Horlein, A. J., et al. 1995. Nature 377:397-404), later studies suggest that they also serve as corepressors to PR (Wagner, B. L., et al. 1998. Mol. Cell. Biol. 18: 1369-1378), ER (Lavinsky, R. M., et al. 1998. Proc. Natl. Acad. Sci. USA 95:2920-2925) and AR (Dotzlaw, H., et al. 2002. Mol. Endocrinol. 16:661-673, Liao, G., et al. 2003. J. Biol. Chem. 278:5052-5061). It is assumed coregulators that can preferentially bind and influence an individual NR at a specific subcellular environment may help to determine the specificity of NR mediated responses.

The p160/steroid receptor coactivator (SRC) family is the most clearly defined class of coactivators, including SRC-1, SRC-2/TIF2, and SRC-3/AIB1/pCIP/RAC3 (Glass, C. K., and M. G. Rosenfeld, Genes Dev 14:121-41 (2000); Llopis, J., et al., Proc Natl Acad Sci USA 97:4363-8 (2000); McKenna, N. J., and B. W. O'Malley, Cell 108:465-74 (2002)). Interaction between ligand-activated steroid receptors and the p160 coactivators is mediated by a small helical motif containing the LXXLL sequence (where L is leucine and X is any amino acid) (44). Ligand binding leads to realignment of the helix 12 in the LBD domain revealing a hydrophobic groove where the LXXLL motifs bind (Bledsoe, R. K., et al., Cell 110: 93-105 (2002), Darimont, B. D., et al., Genes Dev 12:3343-56 (1998), Feng, W., et al., Science 280:1747-9 (1998), Heery, D. M., et al., Nature 387:733-6 (1997)). In addition to LXXLL motifs, a number of AR coregulators, such as ARA54 and ARA70, interact with AR in an androgen-dependent manner through FXXLF motifs (where F is phenylalanine) (He, B., et al., J Biol Chem 277:10226-35 (2002), Kang, H. Y., et al., J Biol Chem 274:8570-6 (1999), 63. Yeh, S., and C. Chang, Proc Natl Acad Sci USA 93:5517-21 (1996)). Furthermore, the FXXLF motif located in the AR N-terminal region is found to mediate the interaction between the LBD and N-terminus of AR (N/C interaction), which is important for the full AR transactivation capacity (Chang, C., J. D. et al., Mol Cell Biol 19:8226-39 (1999), He, B., et al., J Biol Chem 275:22986-94 (2000), Langley, E., et al., J Biol Chem 270:29983-90 (1995)). Phage display technique confirms the FXXLF motif is a ligand-dependent AR associated peptide moti (Hsu, C. L., et al., J Biol Chem 278:23691-8 (2003)). One of the AR coregulators, ARA54, can enhance transactivation of wild-type AR and a mutant AR, derived from LNCaP prostate cancer cells, in prostate cancer cells by 2-6 fold in the presence of androgens or the antiandrogen hydroxyflutamide (HF) (Kang et al. (1999) J. Biol. Chem. 274, 8570-8576; Yeh et al. (1999) Endocrine 11, 195-202).

4. Androgen Receptor Signaling

Upon androgen binding, AR dissociates from the heat-shock proteins and binds to androgen response elements (AREs), resulting in upregulation or downregulation of the transcription of AR target genes. In addition to responding to ligands, the AR is affected by kinase signaling pathways which directly or indirectly alter the biological response to androgens. This phenomenon is mediated by the AR, as antiandrogens have been shown to block kinase-induced transcriptional activation (Sadar, M. D. (1999) J Biol Chem 274 (12), 7777-83). Growth factors, cytokines, and neuropeptides have been implicated in various in vitro and in vivo models of human malignancies, including prostate cancers (Burfeind, P., et al. (1996) Proc Natl Acad Sci USA 93 (14), 7263-8). In the absence of androgens, insulin-like growth factor-1 (IGF-1), keratinocyte growth factor (KGF), and epidermal growth factor (EGF) are able to activate transcription of androgen receptor-regulated genes in prostate cancer cells (Culig, Z., et al. (1995) Eur Urol 27 (Suppl 2), 45-7). MAPK and Akt kinase cascades have been shown to be involved in growth factor-mediated AR activation (Yeh, S., et al. (1999) Proc Natl Acad Sci USA 96 (10), 5458-63, Wen, Y., et al. (2000) Cancer Res 60 (24), 6841-5, Lin, H. K., et al. (2001) Proc Natl Acad Sci USA 98 (13), 7200-5). Some neuropeptides, such as bombesin and neurotensin, can stimulate AR activation and cancer cell growth in the absence of androgen, by activation of tyrosine kinase signaling pathways (Lee, L. F., et al. (2001) Mol Cell Biol 21 (24), 8385-97). Prostate cancer cells may progress from androgen-dependence to a refractory state resulting from activation of AR by various kinases, thus circumventing the normal growth inhibition caused by androgen ablation.

The disclosed methods of treatment or inhibition of uncontrolled cellular proliferation utilize the action of an agent that inhibits one or more activities of androgen or AR. It is understood that such agents can be chemical agents, nucleic acid based agents such as siRNA, inhibitory peptides, or antibodies.

5. Antiandrogens

There are a number of different types of prostate cancer therapies. For example, hormonal secretion from the hypothalamus can be modulated by LH-RH agonists, such as Lupron (Formula 1, Cas Nr 0053714-56-0) 5′ oxo-Pro-His-Trp-Ser-Tyr-Dleu-Leu-Arg-Pro-NH—CH₂—CH₃ and Zoladex, (Formula 2, Cas Nr. 0065807-02-5)

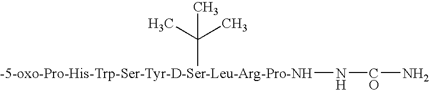

which inhibit the production of T by the testes and adrenal glands. There are also anti-androgen therapeutics, such as Flutamide (Formula 3, 0013311-84-7)

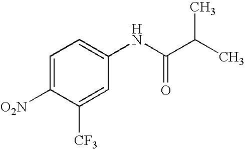

### Formula 3,

### Casodex (Formula 4, Cas Nr. 0090357-06-5)

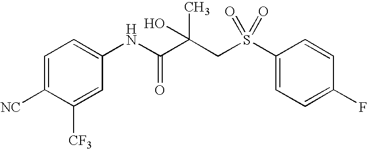

### Formula 4,

### and Nilutamide (Formula 5, Cas Nr. 0063612-50-0)

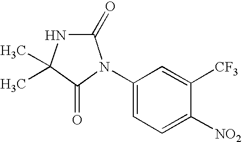

### Formula 5,

which can block the androgen binding to AR. Other therapies include the administration of 5-α reductase inhibitors, such as Proscar (Finasteride) (Formula 6 as Nr. 0098319-26-7)

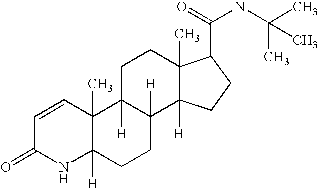

Formula 6, which can inhibit the conversion of T to DHT. DHT is the most effective ligand for AR with higher binding affinity that T. However, this compound is generally applied for BPH patients than for prostate cancer patients.

Thus, disclosed are anti-prostate cancer compounds, such as, flutamide/HF, casodex, niflutamide, finasteride, 1,25-dihydroxyl, vitamin D3, spironolactone, cyproterone acetate, ketoconazole, and natural products including quercetin, resveratrol, silymarin, isoflavonoids, epigallocatechin gallate (EGCG), docosahexaenoic acid (DHA) and eicosapentaenoic acid (EPA). These and others, can all be added in combination with MAP kinase pathway inhibitors, collectively or individually in any combination. It is understood that any of these known anti-prostate cancer compounds and treatments can be used to inhibit uncontrolled cellular proliferation in the bladder or treat bladder cancer using the methods disclosed herein. Thus, for example, specifically disclosed are methods of inhibiting uncontrolled cellular proliferation in the bladder or treating bladder cancer in a subject comprising administering to the subject an agent that inhibits that activity of androgen or AR, wherein the agent is hydroxyflutamide or casodex.

Typically, the anti-prostate cancer compounds can be provided at concentrations of less than or equal to 20 uM, 15 uM, 10, uM, 5 uM, 2 uM, 1 uM, 0.1 uM, or 0.01 uM. Typically the anti-androgens can also be provided at concentrations of less than or equal to 20 uM, 15 uM, 10, uM, 5 uM, 2 uM, 1 uM, 1 uM, or 0.01 uM. Typically the MAP kinase pathway inhibitors, can be administered at concentrations of less than or equal to 100 uM, 90 uM, 80 uM, 70 uM, 60 uM, 50 uM, 40 uM, 30 uM, 20 uM, 15 uM, 10, uM, 5 uM, 2 uM, 1 uM, 0.1 uM, or 0.01 uM. However those of skill in the art understand how to assay for the optimal concentration for administration in vivo, of any of the disclosed compositions, by for example, relying on disclosed cell and animal models for action, as well as by testing the compositions in vivo at various concentrations.

Thus, in accordance with the purpose(s) of these methods, as embodied and broadly described herein, these methods disclosed herein, in one aspect, relates to a method of treating bladder cancer in a subject comprising administering to the subject an effective amount of an agent, wherein the agent is an analog, derivative, or metabolite of curcumin, for example, ASC-J9.

Curcumin [diferuloyl methane; 1,7-bis-(4-hydroxy-3-methoxyphenyl)-1,6-heptadiene-3,5-dione; CR] is the major constituent in the rhizome of Curcuma longa (Zingiberaceae), commonly named turmeric. The structure of curcumin can be divided into 3 parts A, B and C. Part A and C are same: para-phenol with a ortho-methoxyl group. B is a α,β-unsaturated 1,3-diketone.

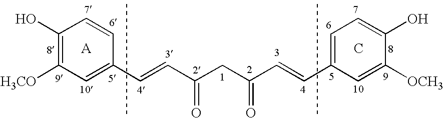

6. Curcumin

Curcumin has been reported to possess various biological and pharmacological activities, including antioxidative (Dutta Sabari et al. Bioorganic & Medicinal Chemistry Letters 15 (2005) 2738-2744; Deng S. L. et al. Food Chemistry 98 (2006) 112-119; Waylon M. Weber, Lucy A. Hunsaker, Steve F. Abcouwer, Lorraine M. Deck, and David L. Vander Jagt, Bioorg. Med. Chem. 13 (2005) 3811-3820; Venkateswarlu S. et al. Bioorganic & Medicinal Chemistry 13 (2005) 6374-6380; Daniel S. et al. J. of Inorganic Biochemistry 98 (2004) 266-275; Priyadarsini K. I. Et al. Free Radical Biology and Medicine, 35 (2003) 475-484), anti-inflammatory (Selvam C. et al. Bioorganic & Medicinal Chemistry Letters 15 (2005) 1793-1797; Chainani-Wu, N. J. of Alternative and Complementary Medicine 9 (2003) 161-168), anti-IIIV-1 integrase (Di Santo R. et al. IL Fannaco 60 (2005) 409-417; Mazumder A. et al. Biochemical Pharmacology, 49 (1995) 1165-1170), anti-angiogenic and anti-cancer (Lin L. et al. Bioorganic & Medicinal Chemistry 14 (2006) 2527-2534; Robinson T. P. et al. Bioorganic & Medicinal Chemistry 13 (2005) 4007-4013; Woo H. B. et al. Bioorganic & Medicinal Chemistry Letters 15 (2005) 3782-3786; Adams B. K. et al. Bioorganic & Medicinal Chemistry 12 (2004) 3871-3883). These putative cancer preventive and therapeutic properties of curcumin have been considered to be associated with its antioxidant and anti-NFKB properties (Deng S. L. et al. Food Chemistry 98 (2006) 112-119; Sabari et al. Bioorganic & Medicinal Chemistry Letters 15 (2005) 2738-2744; Kelly M. R. et al. Mutation Research 485 (2001) 309-318) since the oxidative damage of DNA, lipid layer and cell membrane are believed to be associated with a variety of chronic health problems, such as cancer, inflammatory, neurodegenerative diseases and aging. It is also believed that the antioxidant activity of curcumin is responsible for its free radical scavenging ability.

Structurally, both of curcumin's hydroxyl groups attached to the aromatic rings (A and C parts) and the methylene CH₂ group of the β-1,3-diketone moiety (B part) are responsible for the formation of free radicals and protection of DNA, RNA, lipid and protein molecules. (Jovanovic S. V. et al. J. Am. Chem. Soc. 2001, 123, 3064-3068; Priyadarsini K. I. et al. Free Rad. Biol. Med. 2003, 35, 475).

It is understood that when the agent is targeted to the androgen or androgen receptor gene, tissue specific targeting can be useful to direct treatment or inhibition to a particular tissue and limit the anti-androgen or anti-androgen effect to the tissue where a cancer or uncontrolled cellular proliferation is present. It is understood that there are many ways known in the art to specifically target a tissue. For example, the anti-androgen or anti-androgen receptor agent can be under the control of a tissue specific promoter that limits expression to the particular tissue. Also disclosed would be expressing the anti-androgen or anti-androgen receptor agent in a vector that is targeted specifically to a tissue. Thus, for example, disclosed herein are methods of inhibiting uncontrolled cellular proliferation in bladder tissue or treating bladder cancer in a subject wherein the anti-androgen or anti-androgen receptor agent is operably linked to a bladder specific promoter such as, for example, the uroplakin II promoter. It is further understood that the agent can be provided in the context of a vector. Thus, disclosed herein are methods of inhibiting uncontrolled cellular proliferation or treating cancer in a subject comprising administering to the subject a vector comprising an agent that inhibits one or more activities of androgen or the androgen receptor (AR) operably linked to a bladder specific promoter. It is further understood that any of the disclosed methods and agents can be combined with any of the other methods or agents disclosed herein. Thus, for example, disclosed herein are methods of inhibiting uncontrolled cellular proliferation or treating cancer comprising administering to a subject a vector comprising an agent that inhibits one or more activities of androgen or AR, and further comprising administering to the subject an anti-androgen or anti-AR agent such as hydroxyflutamide (HF), casodex, or ASC-J9. Thus, disclosed herein are methods of treating a bladder cancer comprising administering to a subject a vector encoding an anti-AR siRNA and ASC-J9.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myclomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

Thus, specifically disclosed herein are methods of treating bladder cancer or uncontrolled cellular proliferation in the bladder comprising administering to a subject one or more of the anti-androgen and/or anti-AR agents and/or vectors disclosed herein.

Disclosed are methods of screening a subject for bladder cancer comprising: a) obtaining a tissue sample, and b) assaying for the presence of androgen receptor, wherein the presence of androgen receptor indicates an increased risk of or presence of bladder cancer. Also disclosed are methods of testing. Screening means identifying the presence of a property while testing means determining if a particular property exists. Also disclosed are methods of screening a subject for bladder cancer comprising: a) obtaining a tissue sample, and b) assaying for the presence of androgen receptor mRNA, wherein the presence of androgen receptor indicates an increased risk of or presence of bladder cancer.

It is understood and herein contemplated that the screening can occur in vivo, ex vivo, or in vitro. It is further understood that the tissue sample can comprise a portion of a tissue as little as a single cell. It is further contemplated herein that the tissue sample can be obtained from any source for a tissue such as blood, lavage, or biopsy. It is also contemplated herein that the subject can be any subject wherein the screening for cancer is desirable. Thus, for example, the subject can be a mammal including but not limited to dog, cat, monkey, horse, cow, mouse, rat, guinea pig, human, or non-human primate. It is further contemplated that the subject can be a male.

Disclosed are methods of treating cancer comprising administering to a subject an androgen receptor inhibitor. It is understood and herein contemplated that inhibition of AR can occur through many means such as block the binding site of its ligand. Specifically contemplated herein are methods, wherein the androgen receptor inhibitor reduces nuclear translocation of androgen receptor, wherein the androgen receptor inhibitor comprises ARA67, or fragment thereof, wherein the androgen receptor inhibitor phosphorylates androgen receptor, wherein the androgen receptor inhibitor comprises GSK2B or fragment thereof, wherein the androgen receptor inhibitor reduces an interaction between the N-terminus and C terminus of androgen receptor, wherein the androgen receptor inhibitor comprises hRad9 or fragment thereof, wherein the androgen receptor inhibitor is ARA67, GSK2B, or hRad9, or fragment thereof, wherein the androgen receptor inhibitor interacts with androgen receptor mRNA, wherein the androgen receptor inhibitor is a functional nucleic acid, wherein the androgen receptor inhibitor is an siRNA.

“Obtaining a tissue sample” or “obtain a tissue sample” means to collect a sample of tissue from a subject or measure a tissue in a subject. It is understood and herein contemplated that tissue samples can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, aspiration, tissue swab, spinal tap, magnetic resonance imaging (MRI), Computed Tomography (CT) scan, Positron Emission Tomography (PET) scan, and X-ray (with and without contrast media).

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-androgen or anti-androgen receptor siRNA is disclosed and discussed and a number of modifications that can be made to a number of molecules including the anti-androgen or anti-androgen receptor siRNA are discussed, specifically contemplated is each and every combination and permutation of anti-androgen or anti-androgen receptor siRNA and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Nucleic Acids

There are a variety of molecules disclosed herein that are nucleic acid based, including for example the nucleic acids that encode, for example, androgen, AR, anti-androgen siRNA, and anti-androgen receptor siRNA as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, androgen and androgen receptor as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of anti-androgen and/or anti-androgen receptor or the genomic DNA of anti-androgen and/or anti-androgen receptor siRNA or they can interact with the polypeptide anti-androgen and/or anti-androgen receptor siRNA. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore, disclosed herein are methods of treating bladder cancer in a subject comprising administering to the subject an anti-AR siRNA and/or an anti-androgen siRNA. It is understood that the siRNA can be administering in a vector and that the vector can be specifically targeted to a tissue. It is also contemplated herein that the siRNA can be operably linked to a tissue specific promoter such as the uroplakin II promoter.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

2. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as anti-androgen or anti-androgen receptor siRNA into the cell without degradation and include a promoter yielding expression of the gene in the cells into which it is delivered. In some embodiments the anti-androgen or anti-androgen receptor siRNAs are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans. Thus disclosed herein are vectors comprising an anti-androgen and/or an anti-AR siRNA. It is further understood that the vector can be targeted to a specific tissue, such as, for example, the bladder. It is further understood that the encoded siRNA can be operably linked to a tissue specific promoter, such as, for example, the bladder specific promoter, uroplakin II. It is further understood that disclosed herein are methods of inhibiting cellular proliferation and in particular uncontrolled cellular proliferation in a subject or treating a cancer in a subject comprising administering to the subject one or more of the vectors disclosed herein.

(1) Retroviral Vectors

A retrovirus is an animal virus belonging to the virus family of Retroviridae, including any types, subfamilies, genus, or tropisms. Retroviral vectors, in general, are described by Venna, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pot, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) orhygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Disclosed herein are cells, wherein the cell has a disrupted AR gene, wherein the disrupted gene is produced by action of a recombinase operably linked to a promoter. It is understood and herein contemplated that the cell can be any cell where disruption of the AR gene is desired. For example, specifically contemplated are cell comprising a disrupted AR gene, wherein the cell is an embryonic stem cell, an embryonic germ cell, a bladder cell, a bladder cancer cell, or bladder cancer cell line. It is further contemplated herein that the action of the recombinase is to excise a disruption in a siRNA. Also disclosed are cells wherein the action of the recombinase is to excise a portion or all of the AR gene. It is further contemplated herein that the promoter can be a tissue specific promoter, such as the bladder specific promoter, uroplakin II. Thus, disclosed herein are cells comprising a disrupted AR gene, wherein the disrupted gene is produced by the action of a recombinase operably linked to the bladder tissue specific promoter, uroplaki II.

It is understood that the disrupted AR gene can be any mammalian AR gene. Thus, for example, disclosed herein are cells wherein the AR gene is a murine AR gene. Also disclosed are cells wherein the AR gene is a human AR gene.

It is specifically contemplated herein that the disclosed cells can be used to create a transgenic mammal. Specifically contemplated herein are transgenic mammals, such as a transgenic mouse, comprising a the cells comprising disrupted AR genes disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Also disclose are animals produced by the process of adding to the animal any of the cells disclosed herein.

4. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with androgen and/or androgen receptor such that androgen is inhibited from interacting with AR. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono elonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fe fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol, 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fe), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmami et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti-androgen and anti-androgen receptor antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

5. Peptides

a) Protein Variants

As discussed herein there are numerous variants of the androgen or AR protein and androgen or AR protein that are known and herein contemplated. In addition, to the known functional androgen or AR strain variants there are derivatives of the androgen or AR proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular described variant from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

6. Antioxidants

Generally, antioxidants are compounds that get react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The compositions can contain any antioxidants, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly .alpha.-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

Flavonoids, also known as “phenylchromones,” are naturally occurring, water-soluble compounds which have antioxidant characteristics. Flavonoids are widely distributed in vascular plants and are found in numerous vegetables, fruits and beverages such as tea and wine (particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof and quercetin).

7. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

8. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

9. Compositions with Similar Functions

It is understood that the compositions disclosed herein have certain functions, such as binding androgen receptor. Disclosed herein are certain structural requirements for performing the disclosed functions, and it is understood that there are a variety of structures which can perform the same function which are related to the disclosed structures, and that these structures will ultimately achieve the same result, for example stimulation or inhibition androgen receptor signaling activity.

### D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Results

(1) Androgens and AR in Bladder Carcinogenesis

Previously ARKO mice in which a Cre-lox P system disrupts exon 2 of the AR gene were generated (Yeh et al., Proc. Natl. Acad. Sci. USA, 99:13498-503 (2002)). Exon 2 of the AR gene encodes the second zinc finger of the AR DNA binding domain. The urinary tract (i.e., kidney, renal pelvis, ureter, bladder, and urethra) in both male and female ARKO mice is morphologically similar to that of their wild-type littermates, although male ARKO mice have hypospadias with castration levels [approximately 95% reduction compared with wild-type male littermates (Chang et al., Proc. Natl. Acad. Sci. USA, 101:6876-81 (2004))] of serum testosterone. For the current analysis, ARKO mice and their wild-type littermates were first treated with the strong genotoxic bladder carcinogen BBN at 0.05%. Previous studies showed that wild-type mice given drinking water containing BBN at 0.01-0.05% for 10-20 weeks develop bladder carcinomas by 40 weeks at a high frequency (30-100%) (Bertram and Craig, Eur. J. Cancer, 8:587-94 (1972) and Ohtani et al., Cancer Res., 46:2001-4 (1986)). Some wild-type male mice were then surgically or chemically castrated while some ARKO male mice were administered DHT injections to modify androgen levels.

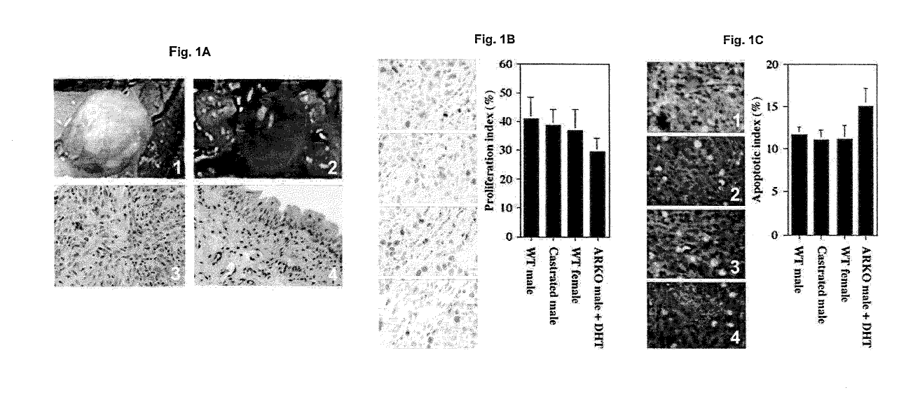

The development of bladder cancer was examined in a total of 168 mice. The mice were divided into seven groups (24 mice/group): (1) male wild-type mice treated with BBN; (2) male castrated wild-type mice treated with BBN; (3) male ARKO mice treated with BBN; (4) male ARKO mice treated with DHT and BBN; (5) female wild-type mice treated with BBN; (6) female ARKO mice treated with BBN; and (7) untreated male wild-type mice. There was no substantial difference in consumption of drinking water with BBN among groups 1 through 6, indicating that intake of BBN was similar. The untreated male wild-type mice did not develop bladder tumors by 40 weeks of age. The incidence of BBN-induced bladder tumors in the other six treatment groups is shown in Table 1, and representative lesions are shown in FIG. 1A. Invasive bladder carcinoma developed in all of the BBN-treated wild-type male mice examined at 30 weeks of age, whereas only 42% of BBN-treated wild-type female mice examined at 40 weeks of age had developed bladder tumors. This finding confirms the difference in incidence of bladder cancer between male and female wild-type mice treated with BBN (Bertram and Craig, Eur. J. Cancer, 8:587-94 (1972)). When examined at 40 weeks, 75% of wild-type male mice treated with BBN had developed preneoplastic lesions (hyperplasias or papillomas). At 3040 weeks, preneoplastic bladder lesions had developed in 50-67% of bladders from the other wild-type mice (castrated males and females). By contrast, at 40 weeks, BBN did not induce any bladder carcinomas in ARKO male or female mice; but rather, there were only two cases (17%) of hyperplasia in the bladder in each group. None of the mice in any group developed renal pelvic, ureteral, or metastatic tumors.

TABLE 1
Incidence of BBN-induced bladder tumors in mice at various time points

| | Age | | | | | | | | |
|---|---|---|---|---|---|---|---|---|---|
| | 20 weeks | | | 30 weeks | | | 40 weeks | | |
| | No. of | Preneoplastic | Carcinoma, | No. of | Preneoplastic | Carcinoma, | No. of | Preneoplastic | Carcinoma, |
| Group | mice | lesions, No. (%) | No. (%) | mice | lesions, No. (%) | No. (%) | mice | lesions, No. (%) | No. (%) |
| WT male | 5† | 2 (40) | 0 (0) | 7 | 5 (71) | 7 (100) | 12 | 9 (75) | 11 (92) |
| Castrated WT male | 3 | 0 (0) | 1 (33) | 8 | 5 (63) | 0 (0)\|\| | 12 | 8 (67) | 6 (50)†† |
| ARKO male | 4 | 0 (0) | 0 (0) | 8 | 0 (0)‡ | 0 (0)\|\| | 12 | 2 (17)# | 0 (0)\|\| |
| ARKO male + DHT | 4 | 0 (0) | 0 (0) | 8 | 3 (38) | 1 (0)∵ | 12 | 5 (42) | 3 (25)‡‡ |
| WT female | 4 | 0 (0) | 0 (0) | 8 | 4 (50) | 0 (0)\|\| | 12 | 18 (67) | 5 (42)§§ |
| ARKO female | 4 | 0 (0) | 0 (0) | 8 | 0 (0)‡, § | 0 (0)\|\| | 12 | 2 (17)#, ** | 0 (0)\|\|, \|\|\|\| |

*BBN = N-butyl-N-(4-hydroxybutyl)nitrosamine; WT = wild-type; ARKO = androgen receptor knockout; DHT = dihydrotestosterone.
†Includes a mouse that died at 24 weeks without a bladder tumor.
‡Statistically significantly different from WT male group by Fisher's exact probability test (p = 0.0070).
§Statistically significantly different from WT female group by Fisher's exact probability test (p = 0.0385).
||Statistically significantly different from WT male group by Fisher's exact probability test (p < 0.001).
¶Statistically significantly different from WT male group by Fisher's exact probability test (p = 0.0012).
#Statistically significantly different from WT male group by Fisher's exact probability test (p = 0.0061).
**Statistically significantly different from WT female group by Fisher's exact probability test (p = 0.0180).
††Statistically significantly different from WT male group by Fisher's exact probability test (p = 0.0343).
‡‡Statistically significantly different from WT male group by Fisher's exact probability test (p = 0.0014).
§§Statistically significantly different from WT male group by Fisher's exact probability test (p = 0.0136).
||||Statistically significantly different from WT female group by Fisher's exact probability test (p = 0.0186).

Together, these results not only confirm the sex differences in BBN-induced bladder cancer incidence but also indicate that the AR plays a role in bladder carcinogenesis. Statistically significant differences in bladder cancer incidence were observed between wild-type male mice and ARKO male mice at 30 (100% vs. 0%, p<0.001) and 40 (92% vs. 0%, p<0.001) weeks and between wild-type female mice and ARKO female mice at 40 weeks (42% vs. 0%, p=0.0186). Female ARKO and female wild-type mice had similar urinary tract characteristics and serum hormone levels [e.g., of testosterone, 17β-estradiol, progesterone, luteinizing hormone, and follicle-stimulating hormone (Shiina et al., Proc. Natl. Acad. Sci. USA, 103:224-9 (2006)). The results disclosed herein indicate that the AR is essential for induction of bladder cancer. Unexpectedly, BBN and DHT treatment of male ARKO mice led to the development of carcinoma and preneoplastic lesions in 25% and 42% of the mice, respectively, when examined at 40 weeks. Additionally, by 40 weeks, 50% of BBN-treated castrated male wild-type mice developed invasive carcinoma. The difference in incidence between DHT-treated and untreated ARKO mice indicates that androgens can be involved in BBN-induced bladder cancer through mechanisms that are independent of the AR. These contrasting findings indicate that androgens (via the AR and non-AR pathways) and the AR (via androgen-mediated and non-androgen-mediated signals) both contribute to bladder carcinogenesis.

(2) Androgens and AR in Bladder Cancer Progression

Having shown that androgens and the AR both influence BBN-induced bladder carcinogenesis, whether either or both also influence bladder cancer progression was next investigated. To do so, both cell proliferation and apoptotic cell death in bladder tumors harvested from mice treated with BBN (groups 1-6) was assessed. The level of cell proliferation was measured by PCNA immunostaining while the level of apoptotic cell death was measured using the TUNEL method. The differences in proliferation and apoptotic indexes between normal epithelia from 11 mice in groups 1 (n=1), 2 (n=2), 3 (n=2), 4 (n=2), 5 (n=2), and 6 (n=2) and invasive bladder carcinomas from 15 mice in groups 1 (n=4), 2 (n=4), 4 (n=3), and 5 (n=4) were significantly different (proliferation index: mean 37.4% vs. mean 1.1%, respectively; difference=36.3%, 95% confidence interval [CI]=28.2 to 44.3, P<0.001 and apoptotic index: mean=9.6% vs. mean=12.4%, difference=2.8%, 95% CI=1.3 to 4.3, P<0.001). There were no differences in the level of proliferation or apoptosis among BBN-induced carcinomas from wild-type male, castrated male, and wild-type female mice (FIGS. 1B and C). Although androgen levels in the mice might also vary, the similar levels of these parameters reflect the similar expression levels of AR as detected by RT-PCR among these tumors. In contrast, BBN-induced bladder tumors in DHT-implanted male ARKO mice had a lower proliferation index [mean=29.6% (ARKO/DHT) vs. 37.4% (others); difference=7.8%; 95% CI=−4.7 to 20.3; P=0.1211] and a higher apoptotic index [mean=15.2% (ARKO/DHT) vs. 12.4% (others); difference=2.8%; 95% CI=0.4 to 5.2; P=0.062] than those from the other three groups (FIGS. 1B and 1C). Cancer progression can correlate with a higher proliferation rate and a lower apoptotic rate, both of which were found in AR-expressing tumors. Thus, these findings indicate that the AR contributes to bladder cancer cell proliferation and apoptosis in BBN-induced bladder cancer, and further indicate that AR activation also promotes bladder cancer progression.

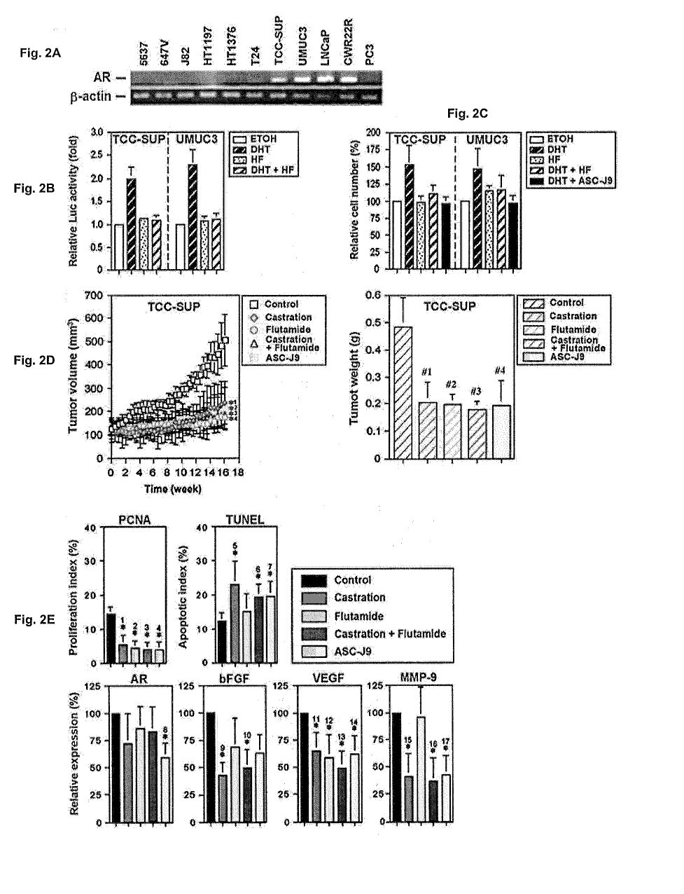

To better understand the role of the AR in the progression of bladder cancer, bladder cancer cell lines that express a functional AR were identified. A recent study used RT-PCR to show that two bladder cancer cell lines, 253J and T24, express the AR (Chen et al., J. Urol., 170:2009-13 (2003)). Therefore, using semi-quantitative RT-PCR, the levels of AR mRNA expression in eight bladder cancer lines, including T24, were determined. This analysis showed AR expression in two of these lines, the human urothelial carcinoma lines TCC-SUP and UMUC3 (FIG. 2A). After 30 cycles of PCR, only a weak AR mRNA signal was detected in the T24 cell line.

By transfecting both lines with the androgen-responsive element-reporter plasmid (MMTV-Luc), the functional activity of the AR in TCC-SUP and UMUC3 cells was next examined. The transfected cells were treated with DHT and/or the antiandrogen HF, and luciferase activity was assayed as a reporter of AR-mediated transcriptional activity. As shown in FIG. 2B, DHT treatment of the TCC-SUP and UMUC3 cell lines increased luciferase activity by 2.0- and 2.3-fold (95% CI=1.7 to 2.3 and 1.9 to 2.7), respectively, over mock treatment. HF alone had only marginal agonist activity by itself but clearly inhibited DHT-induction of luciferase. In contrast, in T24 and other bladder cancer cells with undetectable AR mRNA, DHT exhibited only a marginal effect on AR transactivation, indicating that the AR in T24 cells is not functionally active. Similar results were obtained when MMTV-Luc was replaced with a synthetic ARE-Luc reporter [(ARE)4-Luc]. These results demonstrate that TCC-SUP and UMUC3 cells possess a functional AR.

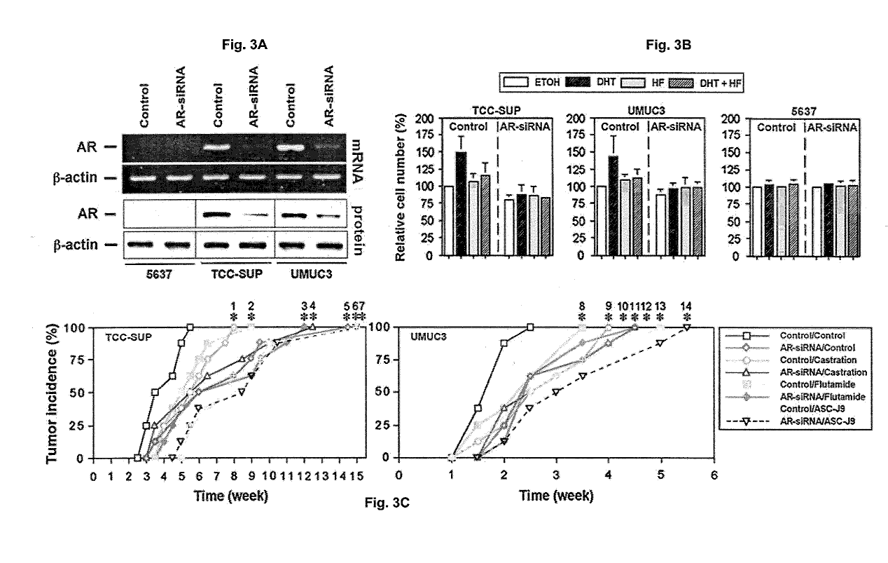

To test the effects of androgen (DHT) and anti androgen (HF) on cell growth of bladder cancer lines, the MTT assay was then utilized. As shown in FIG. 2C, DHT treatment increased the growth of TCC-SUP cells (by 55%, 95% CI=28 to 82%) and UMUC3 cells (by 45%, 95% CI=18 to 73%), while HF treatment antagonized, at least partially, the DHT effect in both lines (67-75% reduction). In contrast, in all other AR-negative bladder cancer cell lines, DHT and HF treatment had only marginal effects on cell growth (<10% changes), see FIG. 3B). These results, together with AR transactivation data, indicate that androgen treatment increases proliferation of AR-positive bladder cancer cells.

(3) Therapeutic Effects on Bladder Cancer Progression of Targeting Androgens or the AR

Based on the finding that androgens transactivate the AR in bladder cancer cells and induce proliferation of AR-positive bladder cancer cells, mouse xenograft models were used to investigate whether targeting androgens and/or the AR suppress bladder cancer progression in vivo. Three therapeutic approaches were employed: (1) androgen deprivation therapy via castration and/or antiandrogen (i.e., flutamide) treatment; (2) treatment with the anti-AR compound ASC-J9; and (3) treatment with an AR siRNA.

(4) Targeting Androgens by Androgen Deprivation Therapy.

Bladder cancer cells (TCC-SUP, UMUC3, and 5637) were implanted subcutaneously into the right and left flanks of 6-week-old male nude mice (n=6 per line). After 2 (for UMUC3) to 4 (for TCC-SUP and 5637) weeks, when the estimated volumes of all tumors for each cell line reached 40 mm³, the mice were either castrated or received a sham surgery, and were then implanted with either slow-releasing flutamide or placebo pellet. Tumor sizes were monitored until the tumors exceeded 10% of the animal's body weight, at which time the mouse was killed (i.e., after 6 to 16 weeks of treatment). As shown in FIG. 2D, TCC-SUP tumors in mice treated by castration and/or with antiandrogen were statistically significantly smaller than those in the control mice at 16 weeks. When the tumors in the treatment groups were harvested, the tumor weights were reduced by 57% to 63%. Similar results (40% to 54% reduction in tumor size in treatment groups at 6 weeks) were obtained in UMUC3 xenograft tumors, whereas minimal effects (up to 12% reduction at 12 weeks) of androgen deprivation therapy were seen in the AR-negative 5637 xenograft tumors.

When the mice were euthanized, tumor specimens were harvested to evaluate cell proliferation (by PCNA immunostaining), apoptosis (by TUNEL assay), and angiogenesis or metastatic ability (expression of bFGF, VEGF, and MMP-9 by real-time RT-PCR). As shown in FIG. 2E, androgen deprivation therapy led to statistically significant (except for an apoptotic index in the flutamide treatment group) decrease in proliferation (62 to 73% reduction) and an increase in apoptosis (19 to 81% induction) in TCC-SUP tumors. This treatment also reduced the levels of angiogenic factors and metastasis-related factors in the tumors. These results indicate that androgen blockade suppresses androgen-sensitive bladder cancer progression.

(5) Targeting AR by Treatment with the Anti-AR Compound ASC-J9.

The recently developed compound ASC-J9 (Ohtsu et al., J. Med. Chem., 45:5037-42 (2002)) directly targets the AR by dissociating AR coregulators from the AR, leading to selective degradation of the AR protein Yang Z et al., Nat. Med. (in press)). The effect of this compound on the growth of AR-positive bladder cancer cells was examined both in vitro and in vivo. As expected, ASC-J9 inhibited DHT-simulated growth of TCC-SUP and UMUC3 cells (FIG. 2C). In the TCC-SUP mouse xenograft model, intraperitoneal injections of ASC-J9 suppressed growth [58% reduction (95% CI=41 to 76%) in tumor size over 16 weeks of treatment] by a similar amount as other androgen deprivation strategies (57-63%; FIG. 2D). Analysis of harvested tumors showed that, like castration and/or treatment with flutamide, treatment with ASC-J9 decreased the proliferation index, increased the apoptotic index, and reduced levels of angiogenic factors and metastasis-related factors (FIG. 2E). In addition, treatment with ASC-J9, but not androgen deprivation, reduced AR expression by 39% (95% CI=28 to 51%). These results indicate that directly targeting the AR can, like targeting androgens, suppress androgen-sensitive bladder cancer progression.

(6) Targeting AR with siRNA.

Stable sublines of TCC-SUP and UMUC3 cells transfected with a retrovirus vector expressing AR-siRNA, which efficiently knocks down the AR in mammalian cells, were first established. (Yeh et al., J. Exp. Med., 198:1899-908 (2003)). As shown in FIG. 3A, levels of AR mRNA and protein were substantially lower in AR-siRNA-expressing TCC-SUP and UMUC3 cells than in cells transfected with scrambled control-siRNA-expressing vector.

The effects of the siRNA-induced reduction in the AR mRNA and protein levels on the proliferation of the stable sublines was next examined. Each stable subline was cultured with DHT and/or HF for 6 days, and cell growth was assessed by MTT assay. In control TCC-SUP or UMUC3 AR-positive cells, DHT induced cell proliferation and HF antagonized the DHT effect (FIG. 3B, see also FIG. 2C). However, AR knockdown by the AR-siRNA resulted in slower cell proliferation [20% reduction (95% CI=12 to 32) in TCC-SUP and 15% reduction (95% CI=6 to 24) in UMUC3] without androgen treatment (FIG. 3B). DHT and HF did not affect the growth of AR-positive TCC-SUP or UMUC3 and AR-negative 5637 sublines that express AR-siRNA

Having found evidence that AR activation promotes BBN-induced mouse bladder cancer development and bladder cancer cell proliferation both in vitro and in vivo, whether the AR signaling in bladder cancer cells influences tumorigenicity was next explored. Stable sublines with AR-siRNA or scrambled control siRNA transfection in mouse xenograft models were used. Each stable subline (TCC-SUP/control-siRNA, TCC-SUP/AR-siRNA, UMUC3/control-siRNA, and UMUC3/AR-siRNA) was injected subcutaneously into the right (control-siRNA-expressing cells) and left (AR-siRNA-expressing cells) flanks of each male nude mouse (n=8 for each treatment group), and treatment (by castration, by flutamide pellet implantation, or with ASC-J9) began immediately. These experiments allowed for the comparison between the development of AR-positive and AR-negative bladder tumor cells, as well as for the evaluation of the effects of androgen deprivation therapy in mice that carry a functional AR gene. As shown in FIG. 3C, AR knockdown (by AR-siRNA expression in tumor cells) or ASC-J9 treatment of the mouse, substantially prolonged the latency of tumor formation, when compared with tumor development of respective cells with control-siRNA expression and control treatment of the host. Androgen deprivation also suppressed tumorigenicity of AR-expressing (control-siRNA-transfected) bladder cancer. By contrast, castration and flutamide treatment had little effect on the development of AR-siRNA-expressing tumors.

### b) Discussion

Previous animal studies utilizing BBN (Bertram and Craig, Eur. J. Cancer, 8:587-94 (1972) and Imada et al., Eur. Urol., 31:360-4 (1997)) showed that this carcinogen induces bladder cancer more easily in males than in females, and that castration retards or reduces the occurrence of BBN-induced bladder cancer. The present findings confirm these earlier studies. Furthermore, bladder carcinomas were found in approximately half of castrated male or wild-type female mice but in none of the male or female mice lacking a functional AR. AR knockout also had a substantial inhibitory effect on premalignant changes in the mouse bladder. These results advance previous observations that used androgen deprivation therapy only (Imada et al., Eur. Urol., 31:360-4 (1997)), and indicate that AR signals can be more important than androgen effects on bladder carcinogenesis.

There was a progressive increase in the incidence of preneoplastic lesions from 30 to 40 weeks in BBN-treated wild-type females and in ARKO males and females, but not in other groups (wild-type males and castrated wild-type males in which ≧50% of mice developed carcinoma at 40 weeks).

Earlier studies have suggested that androgens and the AR might regulate the P450 system (Imaoka et al., Cancer Lett., 166:119-23 (2001)), thereby leading to differential activation of BBN in male and female mice. However, the large difference in BBN-induced bladder cancer incidence between wild-type (42% at 40 weeks) and ARKO (0%) females, both of which have similar levels of sex hormones and morphologically identical bladders, supports the importance of non-P450-involved AR functions in bladder carcinogenesis. Other utilized AR knockdown approaches support this conclusion, i.e., R-siRNA and the anti-AR molecule ASC-J9 in mouse xenograft models, which showed an effect of AR reduction on the growth of non-BBN-induced bladder cancer. Moreover, two bladder cancer cell lines that expressed a functional AR and that AR knockdown, as well as androgen deprivation therapy in male mice, resulted in a substantial delay in tumor formation by these lines.

In the BBN mouse model, the differences in cancer incidence between castrated males (50% at 40 weeks) and ARKO males (0%), and between DHT-supplemented ARKO males (25% at 40 weeks) and ARKO males and females (0%), indicate the involvement of non-androgen-mediated AR signals and androgen-mediated non-AR signals, respectively, in inducing bladder carcinogenesis. Indeed, non-classical AR signal pathways have been reported in prostate cancer (Miyamoto et al., Prostate, 61:332-53 (2004)). For example, peptide growth factors or protein kinases induce AR activity through signal transduction pathways. On the other hand, androgen-mediated non-AR pathways, which might include activation of other steroid hormone receptors, including estrogen receptor, by DHT or its metabolites, have also been suggested (Guerini et al., Cancer Res., 65:5445-53 (2005)). Taken together, this data strongly imply that the AR as well as androgens are targets for controlling bladder cancer development.

Whether androgens regulate bladder cancer progression through the AR was also investigated. Cell proliferation assays and mouse xenograft models revealed that androgen increases the growth of AR-positive bladder cancer cells. Conversely, androgen depletion and/or antiandrogen treatment suppressed cancer progression. It is well known that prostate cancer cells generally require androgens for growth and regress in response to androgen deprivation (Feldman and Feldman, Nat. Rev. Cancer, 1:34-45 (2001) and Miyamoto et al., Prostate, 61:332-53 (2004)). These results indicate that proliferation of some bladder cancers is also androgen sensitive. Furthermore, because AR knockdown in AR-expressing bladder cancer cell lines by siRNA also decreased cell proliferation, even in androgen-depleted conditions, it is likely that AR signals (via androgen-mediated and non-androgen-mediated pathways) contributes to the promotion of bladder cancer progression.

Androgen deprivation reduced the expression of several molecules involved in angiogenesis or metastasis (i.e., bFGF, VEGF, MMP-9) in bladder cancer xenografts and the changes were consistent with xenograft tumor progression or regression. It is important to identify AR-regulated genes in bladder cancer. The identification of such genes leads not only to the elucidation of the role of the AR in bladder cancer, but also to the development of useful markers for detecting bladder cancer and monitoring its recurrence or progression.

These results provide the bases for the development of new preventive or therapeutic approaches for bladder cancer, via targeting androgens and the AR. Several agents and strategies used for treating and preventing prostate cancer, including but not limited to those disclosed herein can be adopted for this purpose and merit consideration for clinical testing (Miyamoto et al., Prostate, 61:332-53 (2004)). More importantly, these findings provide the first evidence indicating the involvement of both androgens (via the AR pathway and non-AR pathway) and the AR (via androgen-mediated signals and non-androgen-mediated signals) in bladder cancer. Targeting both androgens (by castration and/or antiandrogen treatment) and the AR (by siRNA technology or small molecules that degrade AR protein, such as ASC-J9) can achieve maximal inhibitory effects on the development and progression of bladder cancer.

c) Methods

(1) Chemicals and Cell Lines

The materials were supplied from the following vendors: DHT (Sigma), hydroxyflutamide (HF) (Schering), 90-day release pellets (placebo, DHT 1.5 mg/pellet, and flutamide 0.5 mg/pellet) (Innovative Research of America), and ASC-J9 [5-hydroxy-1,7-bis(3,4-dimethoxyphenyl)-1,4,6-heptatrien-3-one] (AndroScience). The human urothelial carcinoma [5637, J82, HT1197, HT1376, T24, TCC-SUP, and UMUC3 from the American Type Culture Collection and 647V originally isolated by Elliott et al., Cancer Res., 36:365-9 (1976)] and the prostatic adenocarcinoma (LNCaP, CWR22R, and PC3) cell lines were maintained in an appropriate medium (Life Technologies; RPMI1640 medium for 5637, LNCaP, and CWR22R; Dulbecco's modified Eagle's medium for J82, HT1197, HT1376, TCC-SUP, UMUC3, 647V, and PC3; McCoy's 5A medium for T24). The media were supplemented with 10% fetal bovine serum (FBS).

(2) Plasmids and Stable Cell Lines

The reporter plasmids [mouse mammary tumor virus (MMTV)-luciferase (Luc) and (ARE)4-Luc (provided by Dr. Michael L. Lu at Harvard Medical School)] as well as a retrovirus vector pMSCV/U6 (Clontech)-AR-siRNA were described in previous studies (Miyamoto et al., Int. J. Cancer, 117:866-72 (2005) and Yeh et al., J. Exp. Med. 198:1899-908 (2003)). Using SuperFect reagent (Qiagen), stable cell lines expressing the AR-siRNA were established by transfecting pMSCV/U6-AR-siRNA into Phi-NX packaging retrovirus producer cells (developed by the Nolan Lab at Stanford University). The target cells (i.e., TCC-SUP, UMUC3, and 5637) were then cultured in the presence of the viral supernatant, and infected cells were selected with puromycin (Sigma).

(3) BBN-Induced Mouse Bladder Cancer Model

Male and female AR knockout (ARKO) mice in the background of the mosaic founder strain C57BL/6-129Sv) were created as previously described (Yeh et al., Proc. Natl. Acad. Sci. USA, 99:13498-503 (2002)). Animal care was provided in accordance with institutional guidelines. Five to six weeks old ARKO mice (male: n=48; female: n=24) and their wild-type littermates (male: n=48; female: n=24) were supplied ad libitum with tap water containing 0.05% BBN (TCI America) in opaque bottles for 12 weeks, and thereafter with tap water without BBN. To estimate BBN intake, the drinking water was prepared fresh twice a week, and consumption was recorded. Negative control mice (wild-type males; n=24) did not receive BBN. Before starting BBN treatment, wild-type male mice received surgical castration (n=24) or sham surgery (n=24) at 5 weeks of age. Similarly, at 5 weeks of age, slow-releasing pellets (DHT 1.5 mg/mouse) were injected into half of the ARKO male mice (n=24). Every 90 days, these pellets were replaced. At 20 (n=4 in each group), 30 (n=8 in each group), or 40 (n=12 in each group) weeks of age, the mice were killed by administration of pentobarbital followed by rapid cervical dislocation. Urinary tract specimens were harvested. These specimens were preserved in phosphate-buffered 10% formalin, embedded in paraffin, sectioned, stained with hematoxylin and eosin, and examined microscopically to identify carcinoma or other pathological changes. Paraffin-embedded sections were also used for immunohistochemical analysis. Part of each specimen was rapidly frozen in liquid nitrogen and stored at −80° C. for subsequent RNA analysis.

(4) Immunohistochemical Analysis of Cell Proliferation and Apoptosis

Sections (5-7 μm thick) from paraffin-embedded mouse urinary tract tissue and xenograft tumors (see below) were deparaffinized in xylene and rehydrated in a graded ethanol series. Sections were then incubated in 3% hydrogen peroxide to block endogenous peroxidase, with a protein blocking solution containing preimmune rabbit serum, and finally with a 1:500 dilution of primary antibody (rabbit polyclonal anti-proliferating cell nuclear antigen (PCNA) (Santa Cruz). Samples were incubated with peroxidase-conjugated anti-rabbit IgG. The slides were rinsed with PBS, incubated with diaminobenzidine, and finally counterstained with hematoxylin. The TUNEL assay was performed on additional rehydrated sections from paraffin-embedded mouse urinary tract tissue and xenograft tumor using the Fluorescein-FragEL DNA Fragmentation Detection kit (Calbiochem), according to the manufacturer's instructions, followed by counterstaining for DNA with DAPI. The percentage of apoptotic cells was determined by fluorescence microscopy. The percentage of PCNA- and TUNEL-positive cells in 1000 cells counted on each specimen were used to determine the amount of cell proliferation and apoptotic indices, respectively. A single observer, who was unaware of the treatment group for the tissue, performed the cell counts.

(5) Reverse-Transcription Polymerase Chain Reaction

Total RNA from mouse bladder and xenograft tumor tissues and cultured cells was extracted by the acid guanidinium-phenol-chloroform method as previously described (Miyamoto et al., Cancer, 75:2565-70 (1995)) or by using Trizol reagent (Life Science). Isolated RNAs were reverse transcribed to cDNA using random hexamers as described (Miyamoto et al., Cancer, 75:2565-70 (1995) and Miyoshi et al., Prostate, 56:280-6 (2003)), and amplified by PCR using a primer set specific for the AR gene (Miyoshi et al., Prostate, 56:280-6 (2003)). Using the iCycler (Bio-Rad) and the methods previously described by Miyoshi et al., Prostate, 56:280-6 (2003), real-time quantitative PCR analysis of AR, basic fibroblast growth factor (bFGF), vascular endothelial growth factor (VEGF), and matrix metalloproteinase-9 (MMP-9) was performed on the tissue samples from mouse xenograft tumors. The following PCR primers were designed using Beacon Designer 2 (Premier Biosoft): bFGF: 5′-AAGAGCGACCCTCACATCAAGCTA-3′ (SEQ ID NO. 1) and 5′-AAGAAACACTCATCCGTAAC-3′ (SEQ ID NO. 2), VEGF: 5′-GGAACACCGACAAACCCA-3′ (SEQ ID NO. 3) and 5′-TCCCCAAAGCACAGCAAT-3′ (SEQ ID NO. 4), and MMP-9: 5′-GGGACGGCAATGCTGATGG-3′ (SEQ ID NO. 5) and 5′-TGGTGGCGCACCAGCGGTAGCCGTC-3′ (SEQ ID NO. 6). β-actin was used as an internal control.

(6) Western Blot of AR Expression

Using the polyclonal AR antibody N20 (Santa Cruz), and previously described methods (Ohtsu H et al., J. Med. Chem., 45:5037-42 (2002)), western blotting analysis was performed in the bladder cancer cell lines. An antibody for β-actin (Santa Cruz) was used as the internal control. Equal amounts of protein (75 μg) obtained from the cell extracts were separated in 10% SDS-polyacrylamide gels and transferred to polyvinylidene diflouride membrane (Millipore) by electroblotting. Specific antibody binding was detected using an alkaline phosphatase detection system (AP-Detection kit, Bio-Rad).

(7) Reporter Gene Assay

Cell transfections and luciferase assays were performed as previously described (Miyamoto et al., Int. J. Cancer, 117:866-72 (2005) and Miyamoto et al., Proc. Natl. Acad. Sci. USA, 100:4440-4 (2003)). Briefly, bladder cancer cells at a density of 50-60% confluence in 12-well tissue culture plates were transfected with 1.5 μg of plasmid DNA using SuperFect transfection reagent (Qiagen) according to the manufacturer's instructions. After 2 or 3 hours, the medium was replaced with medium supplemented with charcoal-stripped FBS in the presence of ligands (testosterone, DHT, HF, and/or ASC-J9) for 24 hours. Cells were harvested, lysed, and assayed for luciferase activity. A Dual-Luciferase Reporter Assay kit (Promega) and luminometer (TD-20/20, Turner BioSystems) were used to determine the level of luciferase activity in the cell extracts.

(8) Cell Proliferation Assay

To assess cell growth, the MTT (thiazolyl blue) assay was used as previously described (Miyamoto et al., Proc. Natl. Acad. Sci. USA, 100:4440-4 (2003). The cells were seeded in 6-well tissue culture plates at a density of 2-5×10⁴ cells/well in medium supplemented with charcoal-stripped FBS containing ligands (testosterone, DHT, HF, and/or ASC-J9). After 6 days of treatment (changing media every 2 days), 200 μL of MTT (Sigma) stock solution (5 mg/mL) was added to each well with 2 mL of medium for 3 hours at 37° C. Then, 1-2 mL of 0.04 N HCl in isopropanol was added to the well. After 5 minutes of incubation at room temperature, the absorbence was measured at a wavelength of 570 nm with background subtraction at 660 nm.

(9) Mouse Xenograft Models

Bladder cancer cell lines (TCC-SUP, UMUC3, and 5637) were harvested, washed twice with PBS, and resuspended in Matrigel (BD Bioscience) at a final concentration of 1×10⁷ cells/mL. Cells (1×10⁶ cells in 100 μl per site) were then injected subcutaneously into the right and left flanks of 6-week-old male athymic nude mouse. Treatment (castration or sham surgery, flutamide [0.5 mg/mouse] or placebo pellet injection, and the recently developed anti-AR molecule ASC-J9 (50 mg/Kg or DMSO injection) (Ohtsu et al., J. Med. Chem., 45:5037-42 (2002)) was initiated either (1) at the same time as tumor cell injection (for tumor incidence studies, n=8 mice in each group) or (2) when the sizes of all tumors reached 40 mm³ (for tumor progression studies, n=12 tumors from 6 mice in each group). Hormonal pellets were replaced as required (every 90 days). Tumor weight was calculated by the following formula: tumor weight (mg)=tumor length (mm)×[tumor width (mm)]²×0.5 (Geram et al., Cancer Chemother. Rep., 3:47-52 (1972)). Using calipers, each tumor was measured twice a week until either the tumor size reached 40 mm³ (for tumor incidence study) (Chen et al., Nat. Med., 10:33-9 (2004)), or until the tumor exceeded 10% of the animal's body weight, at which time the mouse was killed (for tumor progression study).

(10) Statistical Analysis

Differences in tumor incidence among groups in the BBN-induced carcinogenesis study and in the tumorigenicity study in nude mice were analyzed by Fisher's exact test. Differences in mean values (i.e., in tumor size, level of gene expression, and amount of cell proliferation or apoptosis) among different groups were analyzed by two-way ANOVA with repeated measures or Student t-test. P values less than 0.05 were considered to be statistically significant. All statistical tests were two-sided.

### F. REFERENCES

Bertram J S, Craig A W. Specific induction of bladder cancer in mice by butyl-(4-hydroxybutyl)-nitrosamine and the effects of hormonal modifications on the sex difference in response. Eur J Cancer 1972; 8:587-94.
Boorjian S, Ugras S, Mongan N P, Gudas L J, You X, Tickoo S K, et al. Androgen receptor expression is inversely correlated with pathologic tumor stage in bladder cancer. Urology 2004; 64:383-8.
Chang C, Chen Y-T, Yeh S-D, Xu Q, Wang R-S, Guillou F, et al. Infertility with defective spermatogenesis and hypotestosternemia in male mice lacking the androgen receptor in Sertoli cells. Proc Natl Acad Sci USA 2004; 101:6876-81.
Chang C, Kokontis J, Liao S. Molecular cloning of human and rat complementary DNA encoding androgen receptors. Science 1988; 240:324-6.
Chen C D, Welsbie D S, Tran C, Baek S H, Chen R, Vessella R, et al. Molecular determinants of resistance to antiandrogen therapy. Nat Med 2004; 10:33-9.
Chen F, Langenstroer P, Zhang G, Iwamoto Y, See W A. Androgen dependent regulation of bacillus Calmette-guerin induced interleukin-6 expression in human transitional carcinoma cell lines. J Urol 2003; 170:2009-13.
Elliott A Y, Bronson D L, Stein N, Fraley E E. In vitro cultivation of epithelial cells derived from tumors of the human urinary tract. Cancer Res 1976; 36:365-9.
Feldman B J, Feldman D. The development of androgen-independent prostate cancer. Nat Rev Cancer 2001; 1:34-45.
Geran R I, Greenberg N H, Macdonald M M, Schumacher A M, Abbott B J. Protocols for screening chemical agents and natural products against animal tumors and other biological systems (3rd Ed.). Protocol 11. Test evaluation. Cancer Chemother Rep 1972; 3:47-52.
Guerini V, Sau D, Scaccianoce E, Rusmini P, Clana P, Maggi A, et al. The androgen derivative 5α-androstane-3β,17β-diol inhibits prostate cancer cell migration through activation of the estrogen receptor β subtype. Cancer Res 2005; 65:5445-53.
Hartge P, Harvey E B, Linehan W M, Silverman D T, Sullivan J W, Hoover R N. Unexplained excess risk of bladder cancer in men. J Natl Cancer Inst 1990; 82:1036-40.
Heinlein C A, Chang C. Androgen receptor in prostate cancer. Endocr Rev 2004; 25:276-308.
Imada S, Akaza H, Ami Y, Koiso K, Ideyama Y, Takenaka T. Promoting effects and mechanisms of action of androgen in bladder carcinogenesis in male rats. Eur Urol 1997; 31:360-4.
Imaoka S, Yoncda Y, Sugimoto T, Ikemoto S, Hiroi T, Yamamoto K, et al. Androgen regulation of CYP4B1 responsible for mutagenic activation of bladder carcinogens in the rat bladder: detection of CYP4B1 mRNA by competitive reverse transcription-polymerase chain reaction. Cancer Lett 2001; 166:119-23.
Jemal A, Siegel R, Ward E, Murray T, Xu J, Smigal C, et al. Cancer statistics, 2006. CA Cancer J. Clin. 2006; 56:106-30.
Laor E, Schiffman Z J, Braunstein J D, Reid R E, Tolia B M, Koss L G, et al. Androgen receptors in bladder tumors. Urology 1985; 25:161-3.
Miyamoto H, Kubota Y, Shuin T, Torigoe S, Dobashi Y, Hosaka M. Expression of transforming growth factor-beta 1 in human bladder cancer. Cancer 1995; 75:2565-70.
Miyamoto H, Marwah P, Marwah A, Lardy H, Chang, C. 3β-Acetoxyandrost-1,5-diene-17-ethylene ketal functions as a potent antiandrogen with marginal agonist activity. Proc Natl Acad Sci USA 2003; 100:4440-4.
Miyamoto H, Marwah P, Marwah A, Yang Z, Chung C-Y, Altuwaijri S, et al. Identification of steroid derivatives that function as potent antiandrogens. Int J Cancer 2005; 117:866-72.
Miyamoto H, Messing E M, Chang C. Androgen deprivation therapy for prostate cancer: Current status and future prospects. Prostate 2004; 61:332-53.
Miyamoto H, Messing E M, Chang C. Does androgen deprivation improve treatment outcomes in patients with low-risk and intermediate-risk prostate cancer? Nat Clin Prac Oncol 2005; 2:236-7.
Miyoshi Y, Uemura H, Fujinami K, Miyamoto H, Miyoshi Y, Ishiguro H, et al. Expression of AR associated protein 55 (ARA55) and androgen receptor in prostate cancer. Prostate 2003; 56:280-6.
Noronha R F X, Roa B R. Sex hormone receptors in localized and advanced transitional cell carcinoma of urinary tract in humans. Urology 1986; 28:401-3.
Ohtani M, Kakizoe T, Nishio Y, Sato S, Sugimura T, Fukushima S, et al. Sequential changes of mouse bladder epithelium during induction of invasive carcinoma by N-butyl-N(−4-hydroxybutyl)nitrosamine. Cancer Res 1986; 46:2001-4.
Ohtsu H, Xiao Z, Ishida J, Nagai M, Wang H-K, Itokawa H, et al. Antitumor agents. 217. Curcumin analogues as novel androgen receptor antagonists with potential as anti-prostate cancer agents. J Med Chem 2002; 45:5037-42.
Salmi A, Santti R, Gustafsson J-Å, Mäkelä S. Co-localization of androgen receptor with estrogen receptor β in the lower urinary tract of the male rat. J Urol 2001; 166:674-7.
Sanchez-Carbayo M, Cordon-Cardo C. Applications of array technology: Identification of molecular targets in bladder cancer. Br J Cancer 2003; 89:2172-7.
Shen J, Wanibuchi H, Waalkes M P, Salim E I, Kinoshita A, Yoshida K, et al. A comparative study of the sub-chronic toxic effects of three organic arsenical compounds on the urothelium in F344 rats; gender-based differences in response. Toxicol Appl Pharmacol 2006; 210:171-80.
Shiina H, Matsumoto T, Sato T, Igarashi K, Miyamoto J, Takamasa S, et al. Premature ovarian failure in androgen receptor-deficient mice. Proc Natl Acad Sci USA 2006; 103:224-9.
Smith A H, Hopenhayn-Rich C, Bates M N, Goeden H M, Hertz-Picciotto I, Duggan H, et al. Cancer risks from arsenic in drinking water. Environ Health Perspect 1992; 97:259-67.
Smith S C, Oxford G, Theodorescu D. The promise of gene-expression in bladder cancer: A clinician's guide. BJU Int 2005; 95:874-80.
Yang Z, Chang Y-J, Yu I-C, Yeh S, Wu C-C, Miyamoto H, et al. ASC-J9 ameliorates spinal and bulbar muscular atrophy phenotype via degradation of androgen receptor. Nat Med, in press.
Yeh S, Hu Y-C, Wang P-H, Xie C, Xu Q, Tsai M-Y, et al. Abnormal mammary gland development and growth retardation in female mice and MCF7 breast cancer cells lacking androgen receptor. J Exp Med 2003; 198:1899-908.
Yeh S, Tsai M-Y, Xu Q, Mu X-M, Lardy H, Huang K-E, et al. Generation and characterization of androgen receptor knockout (ARKO) mice: An in vivo model for the study of androgen functions in selective tissues. Proc Natl Acad Sci USA 2002; 99:13498-503.
Zhuang Y-H, Bläuer M, Tammela T, Tuohimaa P. Immunodetection of androgen receptor in human urinary bladder cancer. Histopathology 1997; 30:556-62.

### G. Sequences

bFGF

SEQ ID NO. 1

AAGAGCGACCCTCACATCAAGCTA
bFGF

SEQ ID NO. 2

AAGAAACACTCATCCGTAAC
VEGF

SEQ ID NO. 3

GGAACACCGACAAACCCA
VEGF

SEQ ID NO. 4

TCCCCAAAGCACAGCAAT
MMP

SEQ ID NO. 5

GGGACGGCAATGCTGATGG
MMP

SEQ ID NO. 6

TGGTGGCGCACCAGCGGTAGCCGTC
hAR siRNA

SEQ ID NO. 7

GTCGGGCCCTATCCCAGTCCCACTTGCTCGAGCAAGTGGG
hAR siRNA

SEQ ID NO. 8

GCGAATTCAAAAAGCCCTATCCCAGTCCCACTTGCTCGAG
part of AR siRNA

SEQ ID NO: 9

GGGCCCCTGGATGGATAGCTAC
Part of AR siRNA

SEQ ID NO: 10

GTAGCTATCCATCCAGGGGCC
AR siRNA

SEQ ID NO: 11

GGGCCCCTGGATGGATAGCTACCTCGAGGTAGCTATCCATCCAGGGGCC
AR siRNA with poly T after U6 promoter

SEQ ID NO: 12

TTTTTGGGCCCCTGGATGGATAGCTACCTCGAGGTAGCTATCCATCCAGGGGCC

SEQ ID NO: 13

GTCGGGCCCTATCCCAGTCCCACTTGCTCGAGCAAGTGGGACTGGGATAGGGCT
TTTTGAATT-CGC
AR protein sequence (Accession No. NM_000044)

SEQ ID NO: 14

MEVQLGLGRVYPRPPSKTYRGAFQNLFQSVREVIQNPGPRHPEA
ASAAPPGASLLLLQQQQQQQQQQQQQQQQQQQQQETSPRQQQQQQGEDGSPQAHRRGP
TGYLVLDEEQQPSQPQSALECHPERGCVPEPGAAVAASKGLPQQLPAPPDEDDSAAPS
TLSLLGPTFPGLSSCSADLKDILSEASTMQLLQQQQQEAVSEGSSSGRAREASGAPTS
SKDNYLGGTSTISDNAKELCKAVSVSMGLGVEALEHLSPGEQLRGDCMYAPLLGVPPA
VRPTPCAPLAECKGSLLDDSAGKSTEDTAEYSPFKGGYTKGLEGESLGCSGSAAAGSS
GTLELPSTLSLYKSGALDEAAAYQSRDYYNFPLALAGPPPPPPPPHPHARIKLENPLD
YGSAWAAAAAQCRYGDLASLHGAGAAGPGSGSPSAAASSSWHTLFTAEEGQLYGPCGG
GGGGGGGGGGGGGGGGGGGGGGEAGAVAPYGYTRPPQGLAGQESDFTAPDVWYPGGMV
SRVPYPSPTCVKSEMGPWMDSYSGPYGDMRLETARDHVLPIDYYFPPQKTCLICGDEA
SGCHYGALTCGSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPIFLNVLEAIEP
GVVCAGHDNNQPDSFAALLSSLNELGERQLVHVVKWAKALPGFRNLHVDDQMAVIQYS
WMGLMVFAMGWRSFTNVNSRMLYFAPDLVFNEYRMHKSRMYSQCVRMRHLSQEFGWLQ
ITPQEFLCMKALLLFSIIPVDGLKNQKFFDELRMNYIKELDRIIACKRKNPTSCSRRF
YQLTKLLDSVQPIARELHQFTFDLLIKSHMVSVDFPEMMAEIISVQVPKILSGKVKPI
YFHTQ
AR cDNA sequence (Accession No. NM_000044)

SEQ ID NO: 15

| | |
|---|---|
| 1 | cgagatcccg gggagccagc ttgctgggag agcgggacgg tccggagcaa gcccacaggc |
| 61 | agaggaggcg acagagggaa aaagggccga gctagccgct ccagtgctgt acaggagccg |
| 121 | aagggacgca ccacgccagc cccagcccgg ctccagcgac agccaacgcc tcttgcagcg |
| 181 | cggcggcttc gaagccgccg cccggagctg ccctttcctc ttcggtgaag tttttaaaag |
| 241 | ctgctaaaga ctcggaggaa gcaaggaaag tgcctggtag gactgacggc tgcctttgtc |
| 301 | ctcctcctct ccaccccgcc tccccccacc ctgccttccc cccctccccc gtcttctctc |
| 361 | ccgcagctgc ctcagtcggc tactctcagc caacccccct caccaccctt ctccccaccc |
| 421 | gcccccccgc ccccgtcggc ccagcgctgc cagcccgagt ttgcagagag gtaactccct |
| 481 | ttggctgcga gcgggcgagc tagctgcaca ttgcaaagaa ggctcttagg agccaggcga |
| 541 | ctggggagcg gcttcagcac tgcagccacg acccgcctgg ttagaattcc ggcggagaga |
| 601 | accctctgtt ttcccccact ctctctccac ctcctcctgc cttccccacc ccgagtgcgg |
| 661 | agcagagatc aaaagatgaa aaggcagtca ggtcttcagt agccaaaaaa caaaacaaac |
| 721 | aaaaacaaaa aagccgaaat aaaagaaaaa gataataact cagttcttat ttgcacctac |
| 781 | ttcagtggac actgaatttg gaaggtggag gattttgttt ttttctttta agatctgggc |
| 841 | atcttttgaa tctacccttc aagtattaag agacagactg tgagcctagc agggcagatc |
| 901 | ttgtccaccg tgtgtcttct tctgcacgag actttgaggc tgtcagagcg ctttttgcgt |
| 961 | ggttgctccc gcaagtttcc ttctctggag cttcccgcag gtgggcagct agctgcagcg |
| 1021 | actaccgcat catcacagcc tgttgaactc ttctgagcaa gagaagggga ggcggggtaa |
| 1081 | gggaagtagg tggaagattc agccaagctc aaggatggaa gtgcagttag ggctgggaag |
| 1141 | ggtctaccct cggccgccgt ccaagaccta ccgaggagct ttccagaatc tgttccagag |
| 1201 | cgtgcgcgaa gtgatccaga acccgggccc caggcaccca gaggccgcga gcgcagcacc |
| 1261 | tcccggcgcc agtttgctgc tgctgcagca gcagcagcag cagcagcagc agcagcagca |
| 1321 | gcagcagcag cagcagcagc agcagcaaga gactagcccc aggcagcagc agcagcagca |
| 1381 | gggtgaggat ggttctcccc aagcccatcg tagaggcccc acaggctacc tggtcctgga |
| 1441 | tgaggaacag caaccttcac agccgcagtc ggccctggag tgccaccccg agagaggttg |
| 1501 | cgtcccagag cctggagccg ccgtggccgc cagcaagggg ctgccgcagc agctgccagc |
| 1561 | acctccggac gaggatgact cagctgcccc atccacgttg tccctgctgg gccccacttt |
| 1621 | ccccggctta agcagctgct ccgctgacct taaagacatc ctgagcgagg ccagcaccat |
| 1681 | gcaactcctt cagcaacagc agcaggaagc agtatccgaa ggcagcagca gcgggagagc |
| 1741 | gagggaggcc tcgggggctc ccacttcctc caaggacaat tacttagggg gcacttcgac |
| 1801 | catttctgac aacgccaagg agttgtgtaa ggcagtgtcg gtgtccatgg gcctgggtgt |
| 1861 | ggaggcgttg gagcatctga gtccagggga acagcttcgg ggggattgca tgtacgcccc |
| 1921 | acttttggga gttccacccg ctgtgcgtcc cactccttgt gccccattgg ccgaatgcaa |
| 1981 | aggttctctg ctagacgaca gcgcaggcaa gagcactgaa gatactgctg agtattcccc |
| 2041 | tttcaaggga ggttacacca aagggctaga aggcgagagc ctaggctgct ctggcagcgc |
| 2101 | tgcagcaggg agctccggga cacttgaact gccgtctacc ctgtctctct acaagtccgg |
| 2161 | agcactggac gaggcagctg cgtaccagag tcgcgactac tacaactttc cactggctct |
| 2221 | ggccggaccg ccgccccctc cgccgcctcc ccatccccac gctcgcatca agctggagaa |
| 2281 | cccgctggac tacggcagcg cctgggcggc tgcggcggcg cagtgccgct atggggacct |
| 2341 | ggcgagcctg catggcgcgg gtgcagcggg acccggttct gggtcaccct cagccgccgc |
| 2401 | ttcctcatcc tggcacactc tcttcacagc cgaagaaggc cagttgtatg gaccgtgtgg |
| 2461 | tggtggtggg ggtggtggcg gcggcggcgg cggcggcggc ggcggcggcg gcggcggcgg |
| 2521 | cggcggcggc gaggcgggag ctgtagcccc ctacggctac actcggcccc ctcaggggct |
| 2581 | ggcgggccag gaaagcgact tcaccgcacc tgatgtgtgg taccctggcg gcatggtgag |
| 2641 | cagagtgccc tatcccagtc ccacttgtgt caaaagcgaa atgggcccct ggatggatag |
| 2701 | ctactccgga ccttacgggg acatgcgttt ggagactgcc agggaccatg ttttgcccat |
| 2761 | tgactattac tttccacccc agaagacctg cctgatctgt ggagatgaag cttctgggtg |
| 2821 | tcactatgga gctctcacat gtggaagctg caaggtcttc ttcaaaagag ccgctgaagg |
| 2881 | gaaacagaag tacctgtgcg ccagcagaaa tgattgcact attgataaat tccgaaggaa |
| 2941 | aaattgtcca tcttgtcgtc ttcggaaatg ttatgaagca gggatgactc tgggagcccg |
| 3001 | gaagctgaag aaacttggta atctgaaact acaggaggaa ggagaggctt ccagcaccac |
| 3061 | cagccccact gaggagacaa cccagaagct gacagtgtca cacattgaag gctatgaatg |
| 3121 | tcagcccatc tttctgaatg tcctggaagc cattgagcca ggtgtagtgt gtgctggaca |
| 3181 | cgacaacaac cagcccgact cctttgcagc cttgctctct agcctcaatg aactgggaga |
| 3241 | gagacagctt gtacacgtgg tcaagtgggc caaggccttg cctggcttcc gcaacttaca |
| 3301 | cgtggacgac cagatggctg tcattcagta ctcctggatg gggctcatgg tgtttgccat |
| 3361 | gggctggcga tccttcacca atgtcaactc caggatgctc tacttcgccc ctgatctggt |
| 3421 | tttcaatgag taccgcatgc acaagtcccg gatgtacagc cagtgtgtcc gaatgaggca |
| 3481 | cctctctcaa gagtttggat ggctccaaat caccccccag gaattcctgt gcatgaaagc |
| 3541 | actgctactc ttcagcatta ttccagtgga tgggctgaaa aatcaaaaat tctttgatga |
| 3601 | acttcgaatg aactacatca aggaactcga tcgtatcatt gcatgcaaaa gaaaaaatcc |
| 3661 | cacatcctgc tcaagacgct tctaccagct caccaagctc ctggactccg tgcagcctat |
| 3721 | tgcgagagag ctgcatcagt tcacttttga cctgctaatc aagtcacaca tggtgagcgt |
| 3781 | ggactttccg gaaatgatgg cagagatcat ctctgtgcaa gtgcccaaga tcctttctgg |
| 3841 | gaaagtcaag cccatctatt tccacaccca gtgaagcatt ggaaacccta tttccccacc |
| 3901 | ccagctcatg ccccctttca gatgtcttct gcctgttata actctgcact actcctctgc |
| 3961 | agtgccttgg ggaatttcct ctattgatgt acagtctgtc atgaacatgt tcctgaattc |
| 4021 | tatttgctgg gctttttttt tctctttctc tcctttcttt ttcttcttcc ctccctatct |
| 4081 | aaccctccca tggcaccttc agactttgct tcccattgtg gctcctatct gtgttttgaa |
| 4141 | tggtgttgta tgcctttaaa tctgtgatga tcctcatatg gcccagtgtc aagttgtgct |
| 4201 | tgtttacagc actactctgt gccagccaca caaacgttta cttatcttat gccacgggaa |
| 4261 | gtttagagag ctaagattat ctggggaaat caaaacaaaa aacaagcaaa caaaaaaaaa |
| 4321 | a |

What is claimed is:

1. A method of inhibiting cellular proliferation in a subject comprising administering to the subject a vector comprising an agent that inhibits one or more activities of androgen or androgen receptor (AR) gene operably linked to a bladder specific promoter.

2. A method of treating a cancer in a subject comprising administering to the subject a vector comprising an agent that inhibits one or more activities of an androgen or androgen receptor gene operably linked to an bladder specific promoter.

3. The method of claims 1 or 2, wherein the cancer is bladder cancer.

4. The method of claims 1 or 2, wherein the bladder specific promoter is the uroplakin II promoter.

5. The method of claims 1 or 2, wherein the agent is an anti-androgen or anti-androgen receptor (AR) small interfering RNA (siRNA).

6. The method of claims 1 or 2, further comprising administering to the subject an anti-androgen or anti-androgen receptor agent.

7. A method of treating bladder cancer in a subject comprising administering to the subject an anti-androgen or anti-androgen receptor agent.

8. The method of claim 7, wherein the agent is an anti-androgen or anti-androgen receptor antibody.

9. The method of claim 7, wherein the agent is an anti-androgen or anti-androgen receptor siRNA.

10. The method of claim 9, wherein the siRNA comprises the sequence set forth in SEQ ID NO: 7-13.

11. The method of claim 7, wherein the agent is a curcumin derivative.

12. The method of claim 11, wherein the curcumin derivative is ASC-J9.

13. The method of claim 7, wherein the agent is hydroxyflutamide.

14. The method of claim 7, wherein the agent is casodex.

15. A vector comprising an anti-androgen or anti-androgen receptor siRNA operably linked to a bladder specific promoter.

16. A method of inhibiting cellular proliferation comprising administering to a subject the vector of claim 15.

17. A method of treating a cancer comprising administering to a subject the vector of claim 15.

18. The method of claim 17, wherein the cancer is bladder cancer.

19. A method of screening a subject for the risk of contracting bladder cancer comprising: a) obtaining a tissue sample, and b) assaying for the presence of androgen receptor or androgen receptor mRNA, wherein the presence of androgen receptor indicates an increased risk of or presence of bladder cancer.

20. A method of screening for an agent that inhibits bladder cancer growth or androgen dependent tumor growth comprising administering the agent to a bladder cell and monitoring the level of androgen receptor activity in the cell, wherein a decrease in androgen receptor activity relative to a control indicates an agent that inhibits bladder cancer growth.

21. A method of screening for an agent that inhibits androgen dependent tumor growth comprising administering the agent to a bladder cell and monitoring the level of androgen receptor activity in the cell, wherein a decrease in androgen receptor activity relative to a control indicates an agent that inhibits androgen dependent tumor growth.

22. A cell, wherein the cell has a disrupted AR gene, wherein the disrupted gene is produced by action of a recombinase operably linked to a promoter.

23. A transgenic mammal comprising the cell of claims 22.

24. The transgenic mammal of claim 23, wherein the mammal is a mouse.
